SEQUENCE LISTING (5 sequences)

SEQ ID NO: 1 - protein, 1665 aa, Artificial sequence
  Synthetic polypeptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 1438 aa, Artificial sequence
  Synthetic polypeptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 227 aa, Artificial sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 226 aa, Artificial sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 1664 aa, Artificial sequence
  Synthetic polypeptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0233650 A1
(43) Pub. Date: Jul. 28, 2022

(54) RECOMBINANT FACTOR VIII-FC FOR TREATING HEMOPHILIA AND LOW BONE MINERAL DENSITY

(71) Applicant: BIOVERATIV THERAPEUTICS INC., Waltham, MA (US)

(72) Inventors: Susu DUAN, Groton, CT (US); Katalin KIS-TOTH, Wellesley, MA (US); Gaurav Manohar RAJANI, Waltham, MA (US); Joe SALAS, Brookline, MA (US)

(21) Appl. No.: 17/618,808

(22) PCT Filed: Jun. 18, 2020
(86) PCT No.: PCT/US2020/038444

§ 371 Date: Dec. 13, 2021

Related U.S. Application Data
(60) Provisional application No. 62/968,785, filed on Jan. 31, 2020.
(60) Provisional application No. 62/863,831, filed on Jun. 19, 2019.

(51) Int. Cl. A61K 38/37; A61K 47/68; A61P 19/08

(52) U.S. Cl. CPC A61K 38/37; A61P 19/08; A61K 47/6811

(57) ABSTRACT

Disclosed herein are methods of treating subjects with hemophilia and low bone mineral density (BMD) with a chimeric protein comprising a coagulation factor and a Fc domain. In certain embodiments, the chimeric protein is rFVIIIFc. In certain embodiments, a subject to be treated has hemophilia A.

### RELATED APPLICATIONS

This application claims the benefit of priority to U.S. Provisional Application No. 62/863,831, filed Jun. 19, 2019, and U.S. Provisional Application No. 62/968,785, filed Jan. 31, 2020, both of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 16, 2020, is named 706564_SA9-503PC_ST25.txt and is 46,080 bytes in size.

### BACKGROUND OF THE DISCLOSURE

Hemophilia is a group of bleeding disorders caused by defects in the genes encoding coagulation factors and affects 1-2 in 10,000 male births. Graw et al., Nat. Rev. Genet. 6(6): 488-501 (2005). Hemophilia A is characterized by the absence of functional endogenous coagulation factor VIII (FVIII). Patients with severe hemophilia A suffer not only from poorly-controlled traumatic bleeds but also from spontaneous bleeding into the joints. The current standard of care for treatment of hemophilia is intravenous factor replacement therapy with the aim of preventing serious life- and limb-threatening bleeding including recurrent joint hemorrhage (hemarthrosis) which could lead to hypertrophic synovitis and cartilage degradation (hemophilic arthropathy). Manco-Johnson et al, NEJM 357(6):535-4 (2007). Over decades, optimal prophylaxis reduces but does not eliminate joint bleeding. Manco-Johnson at al, Blood 129(17):2368-2374 (2017).

People with hemophilia are at higher risk for reduced bone mineral density (BMD) and osteoporosis compared to the general population. Gerstner et al, Haemophilia, 15(2):559-65 (2009). According to one study, 27% of hemophiliacs have osteoporosis and 43% have low bone density. Id. Growing global observations of BMD indicate it is often lower in hemophilia patients than control cases or lower than expected in general populations based on age. Despite this association, the mechanism of reduced BMD in hemophilia patients is currently unknown.

A significant reduction in both lumbar spine and hip BMD of hemophilia patients begins in childhood. There is a need for improved treatment options for hemophilia patients that protect against joint bleeds and minimize loss of BMD over time.

### SUMMARY

Provided herein are, inter alia, methods and compositions for treating subjects with hemophilia and low BMD. Certain aspects of the present disclosure are directed to a method of treating a subject with hemophilia A and low bone mineral density (BMD), the method comprising selecting a subject having hemophilia A and low BMD, and administering to the subject a therapeutically effective amount of a chimeric protein comprising a recombinant Factor VIII (FVIII) protein and a Fc domain (rFVII1Fc), wherein administration of the chimeric protein inhibits reduction of BMD in the subject. In some embodiments, the Fc domain is the Fc domain of immunoglobulin G1 (IgG1). In some embodiments, the Fc domain is the Fc domain of human IgG1. In some embodiments, the chimeric protein is rFVII1Fc. Some aspects of the present disclosure are directed to a chimeric protein comprising a recombinant FVIII protein and a Fc domain for use in treating a subject with hemophilia A and low bone mineral density (BMD).

In some embodiments, the subject has mild hemophilia A. In some embodiments, the subject has moderate hemophilia A. In some embodiments, the subject has severe hemophilia A.

In some embodiments, the rFVII1Fc comprises an amino acid sequence at least 95% identical to an amino acid sequence according to SEQ ID NO: 1. In some embodiments, the rFVII1Fc comprises an amino acid sequence according to SEQ ID NO: 1.

In some embodiments, the FVIII portion of the chimeric protein comprises an amino acid sequence at least 95% identical to an amino acid sequence according to SEQ ID NO: 2. In some embodiments, the FVIII portion of the chimeric protein comprises an amino acid sequence according to SEQ ID NO: 2.

In some embodiments, the rFVII1Fc comprises an amino acid sequence at least 95% identical to SEQ ID NO: 5. In some embodiments, the rFVII1Fc comprises an amino acid sequence identical to SEQ ID NO: 5.

In some embodiments, the chimeric protein comprises a first polypeptide chain comprising an amino acid sequence at least 95% identical to SEQ ID NO: 5 and a second polypeptide chain comprising an amino acid sequence at least 95% identical to SEQ ID NO: 4. In some embodiments, the chimeric protein comprises a first polypeptide chain comprising an amino acid sequence identical to SEQ ID NO: 5 and a second polypeptide chain comprising an amino acid sequence identical to SEQ ID NO: 4. In some embodiments, the chimeric protein comprises a first polypeptide chain whose amino acid sequence is identical to SEQ ID NO: 5 and a second polypeptide chain whose amino acid sequence is identical to SEQ ID NO: 4. In some embodiments, the first polypeptide chain is covalently bound to the second polypeptide chain via a disulfide bond. In some embodiments, the chimeric protein comprises a first polypeptide chain that is covalently bound to a second polypeptide chain via two disulfide bonds. In some embodiments, the chimeric protein comprises a first polypeptide chain that is covalently bound to a second polypeptide chain via two disulfide bonds in a hinge region of the Fc domain. In some embodiments, the chimeric protein is efmoroctocog alfa. In some embodiments, the efmoroctocog alfa is sold under the tradename ELOCTA® or ELOCTATE® or is a biosimilar thereof.

In some embodiments, the chimeric protein comprises a first polypeptide chain that is covalently bound to a second polypeptide chain via two disulfide bonds in a hinge region of the Fc domain, wherein the first polypeptide chain comprises a first polypeptide chain whose amino acid sequence is identical to SEQ ID NO: 5 comprising sulfated tyrosines at Y346, Y718, Y719, Y723, Y770, and Y786, N-glycosylation sites at N41, N239, N916, N1224 and N1515 and a second polypeptide chain whose amino acid sequence is identical to SEQ ID NO: 4 comprising an N-glycosylation site at N77.

In some embodiments, the method comprises administering to the subject an effective amount of a pharmaceutical composition comprising (i) a chimeric polypeptide, which comprises a FVIII protein and an Fc domain, and (ii) at least one pharmaceutically acceptable excipient, wherein about 1% to about 40% of the FVIII protein of the chimeric polypeptide is single-chain FVIII and about 60% to about 99% of the FVIII protein of the chimeric polypeptide is processed FVIII, wherein the single-chain FVIII protein comprises a FVIII heavy chain and a FVIII light chain on a single polypeptide chain, and the processed FVIII comprises a FVIII heavy chain and a FVIII light chain on two polypeptide chains.

In some embodiments, the chimeric protein has been produced by human cells. In some embodiments, the human cells are human embryonic kidney 293 (HEK293) cells. In some embodiments, the human cells are HEK293F cells.

In some embodiments, the rFVII1Fc is administered at a dose of 25-65 IU/kg every 3-5 days. In some embodiments, the recombinant FVIII protein is administered at a dose of 25-65 IU/kg every 3 days. In some embodiments, the recombinant FVIII protein is administered at a dose of 25-65 IU/kg every 4 days. In some embodiments, the recombinant FVIII protein is administered at a dose of 25-65 IU/kg every 5 days.

In some embodiments, the subject is 50 years of age or older. In certain embodiments, the subject is younger than 50 years of age.

In some embodiments, BMD in the subject is measured by X-Ray. In some embodiments, BMD in the subject is measured by Dual X-Ray Absorptiometry (DXA).

In some embodiments, a subject with low BMD has osteopenia and/or osteoporosis. In some embodiments, a subject with low BMD has osteopenia. In some embodiments, a subject with low BMD has osteoporosis. In some embodiments, BMD in the subject is determined by T-score. In some embodiments, the subject is determined to have low BMD if the subject has a T-score of less than −1.0. In some embodiments, the subject is determined to have low BMD and osteopenia if the subject has T-score between −1.0 and −2.4. In some embodiments, the subject is determined to have low BMD and osteoporosis if the subject has a T-score of less than or equal to −2.5.

In some embodiments, BMD in the subject is determined by Z-score. In some embodiments, the subject is determined to have low BMD if the subject has a Z-score of less than −2.0.

In some embodiments, the subject is predicted to have low BMD based on the level of one or more biomarkers of bone formation, bone resorption, and/or bone loss. In some embodiments, the biomarker is assessed (e.g., the level or amount of the protein is measured with an assay) from the peripheral blood or urine of the subject. In some embodiments, the level of one or more biomarkers is measured in a biological sample that is peripheral blood or is derived from peripheral blood (such as serum or plasma). In some embodiments, the one or more biomarkers of bone formation comprise bone-specific alkaline phosphatase, procollagen type 1 N-terminal propeptide (P1NP), procollagen type 1 C-terminal propeptide (P1CP), and/or osteocalcin. In some embodiments, the one or more biomarkers of bone resorption comprise total alkaline phosphatase in serum, the receptor activator of nuclear factor kappa B (RANKL), osteoprotegerin (OPG), tartrate-resistant acid phosphatase (TRAP), hydroxylysine, hydroxyproline, deoxypyridinoline (DPD), pyridinoline (PYD), bone sialoprotein, cathepsin K, tartrate-resistant acid phosphatase 5b (TRAP5b), matrix metalloproteinase 9 (MMP9), and/or C- and N-terminal cross-linked telopeptide for type 1 collagen (CTX-1 and NTX-1, respectively).

In some embodiments, the subject does not have a vitamin D deficiency. In some embodiments, the subject has been previously treated with a Factor VIII without an Fc portion.

Certain aspects of the present disclosure are directed to a method of treating a subject with hemophilia A and an increased risk of bone fracture, the method comprising selecting a subject having hemophilia and an increased risk of bone fracture, and administering to the subject a therapeutically effective amount of a chimeric protein comprising a recombinant FVIII protein and a Fc domain, wherein administration of the chimeric protein reduces the risk of bone fracture in the subject. Some aspects of the present disclosure are directed to a chimeric protein comprising a recombinant FVIII protein and a Fc domain for use in treating a subject with hemophilia A and an increased risk of bone fracture.

In some embodiments, the risk of bone fracture in the subject is determined by the fracture risk assessment tool (FRAX). In some embodiments, the risk of bone fracture in the subject is determined by assessment of low BMD risk factors. In some embodiments, the low BMD risk factors comprise arthropathy, reduced physical activity, infection with HIV or HCV, vitamin D deficiency, low body mass index (BMI), and/or hypogonadism.

Certain aspects of the present disclosure are directed to a method of treating a subject with hemophilia A and a bone fracture, the method comprising selecting a subject having hemophilia and a bone fracture, and administering to the subject a therapeutically effective amount of a chimeric protein comprising a recombinant FVIII protein and a Fc domain. Some aspects of the present disclosure are directed to a chimeric protein comprising a recombinant FVIII protein and a Fc domain for use in treating a subject with hemophilia A and a bone fracture.

Certain aspects of the present disclosure are directed to a method of reducing the rate of bone mineral density (BMD) loss in a subject, the method comprising selecting a subject with low BMD; and administering to the subject a therapeutically effective amount of a chimeric protein comprising a coagulation factor and a Fc domain, such that administration of the chimeric protein reduces the rate of BMD loss in the subject. Some aspects of the present disclosure are directed to a chimeric protein comprising a recombinant FVIII protein and a Fc domain (rFVII1Fc) for use in treating a subject with hemophilia A and reducing the rate of BMD loss in the subject.

Certain aspects of the present disclosure are directed to a method of increasing bone mineral density (BMD) and prophylactically treating bleeding episodes in a subject who has hemophilia A, the method comprising: (i) identifying a subject who is receiving treatment for hemophilia A with a FVIII protein without an Fc portion, wherein the subject has had adequate blood clotting during the treatment, and wherein the subject has low BMD; (ii) discontinuing treatment with the FVIII protein without an Fc portion and administering to the subject a therapeutically effective amount of a chimeric protein comprising a recombinant FVIII protein and a Fc domain (rFVII1Fc), wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject who has hemophilia A, the method comprising: (i) identifying a subject who is receiving treatment for hemophilia A with a non-factor replacement protein, wherein the subject has had adequate blood clotting during the treatment, and wherein the subject has low BMD; (ii) discontinuing treatment with the non-factor replacement protein and administering to the subject a therapeutically effective amount of a chimeric protein comprising a recombinant FVIII protein and a Fc domain (rFVII1Fc), wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and low BMD, and wherein administration of the chimeric protein increases BMD and prophylactically treats bleeding episodes in the subject.

Certain aspects of the present disclosure are directed to a method of reducing the risk of fracture and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and an increased risk of fracture, and wherein administration of the chimeric protein reduces the risk of fracture and prophylactically treats bleeding episodes in the subject.

Certain aspects of the present disclosure are directed to a method of reducing the rate of bone mineral density (BMD) loss and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and BMD loss, and wherein administration of the chimeric protein reduces the rate of BMD loss and prophylactically treats bleeding episodes in the subject.

Certain aspects of the present disclosure are directed to a method of increasing bone mineral density (BMD) and prophylactically treating bleeding episodes in a subject who has hemophilia A and is being treated with a FVIII protein without an Fc portion, the method comprising discontinuing treatment with the FVIII protein without an Fc portion and administering to the subject a therapeutically effective amount of a chimeric protein comprising a recombinant FVIII protein and a Fc domain (rFVII1Fc), wherein the subject has been identified as having low BMD and adequate blood clotting during treatment with the FVIII protein without an Fc portion, and wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject who has hemophilia A and is being treated with a non-factor replacement protein, the method comprising discontinuing treatment with the non-factor replacement protein and administering to the subject a therapeutically effective amount of a chimeric protein comprising a recombinant FVIII protein and a Fc domain (rFVII1Fc), wherein the subject has been identified as having low BMD and adequate blood clotting during treatment with the non-factor replacement protein, and wherein administration of the chimeric protein increases BMD and prophylactically treats bleeding episodes in the subject.

In some embodiments, the subject has been previously treated to reduce bleeding associated with hemophilia A using a Factor VIII protein without an Fc portion.

In some embodiments, the Factor VIII protein without an Fc portion is PEGylated FVIII that is not fused to a Fc domain.

In some embodiments, the Factor VIII protein without an Fc portion is single-chain FVIII that is not fused to a Fc domain.

In some embodiments, the Factor VIII protein without an Fc portion is recombinant FVIII that does not comprise a moiety that extends the half-life thereof in humans.

In some embodiments, the Factor VIII protein without an Fc portion is blood-derived FVIII or plasma-derived FVIII.

In some embodiments, the Factor VIII protein without an Fc portion is damoctocog alfa pegol, turoctocog alfa pegol, turoctocog alfa, lonoctocog alfa, simoctocog alfa, rurioctocog alfa pegol, or octocog alfa.

In some embodiments, the subject has been previously treated to reduce bleeding associated with hemophilia A using a non-factor replacement protein.

In some embodiments, the non-factor replacement protein is emicizumab.

In some embodiments, the emicizumab is emicizumab-kxwh.

In some embodiments, the subject had adequate blood clotting during treatment with the Factor VIII protein without an Fc portion or the non-factor replacement protein.

In some embodiments, the subject has low BMD at a bone site and/or joint where bleeding has not been detected.

In accordance with each of the foregoing aspects and embodiments, in certain embodiments, the subject has mild hemophilia A. Alternatively, in accordance with each of the foregoing aspects and embodiments, in certain embodiments, the subject has moderate hemophilia A. Alternatively, in accordance with each of the foregoing aspects and embodiments, in certain embodiments, the subject has severe hemophilia A.

### BRIEF DESCRIPTION OF THE DRAWINGS

FIGS. 1A-B are schematic representations of an experimental in vitro model using monocyte-derived cell types to examine macrophage and osteoclast morphology by tartrate-resistant acid phosphatase (TRAP) staining, osteoclast bone resorption activity, gene expression profiling, osteoclast-specific genes, and antioxidation pathway-associated genes. FIG. 1A is a schematic displaying the protocol for differentiating CD14⁺ monocytes to monocyte-derived macrophages by administering M-CSF alone at 50 ng/ml over 7 days. FIG. 1B is a schematic displaying the protocol for differentiating CD14⁺ monocytes to monocyte-derived osteoclasts by administering M-CSF at 50 ng/ml and RANKL 100 ng/ml over 7 days.

FIGS. 2A-B are schematic representations of an experimental in vitro model using monocyte-derived cell types to examine tartrate-resistant acid phosphatase (TRAP) staining. FIG. 2A is a schematic displaying the control group of CD14⁺ monocytes differentiated to monocyte-derived macrophages by administering macrophage colony-stimulating factor (M-CSF) alone at 50 ng/mlover 7 days and examined for TRAP staining. FIG. 2B is a schematic displaying the test groups of CD14⁺ monocytes differentiated to monocyte-derived osteoclasts by administering M-CSF at 50 ng/ml and RANKL 100 ng/ml over 7 days and treated at day 0 with Vehicle, IgG1 (25 nM), rFVIII (25 nM), or rFVII1Fc (25 nM).

FIGS. 3A-E are visual depictions of TRAP staining in monocyte-derived macrophages (FIG. 3A) and monocyte-derived osteoclasts (FIGS. 3B-3E). FIG. 3B is a visual depiction of TRAP staining in monocyte-derived osteoclasts treated with vehicle. FIG. 3C is a visual depiction of TRAP staining in monocyte-derived osteoclasts treated with IgG1 alone. FIG. 3D is a visual depiction of TRAP staining in monocyte-derived osteoclasts treated with recombinant factor VIII (rFVIII) alone. FIG. 3E is a visual depiction of TRAP staining in monocyte-derived osteoclasts treated with rFVIIIFc.

FIG. 4 is a schematic representation of a washout experiment to determine osteoclast formation in which CD14⁺ monocytes are treated for one day prior to differentiation into monocyte-derived osteoclasts with one of 4 treatments: Vehicle treatment, IgG1 alone, rFVIII, or rFVII1Fc. Cells were analyzed for morphology at day 7.

FIGS. 5A-D are visual depictions of monocyte-derived osteoclast morphology 7 days after differentiation when treated for one day prior to differentiation with vehicle (FIG. 5A), IgG1 (FIG. 5B), rFVIII (FIG. 5C), or rFVII1Fc (FIG. 5D).

FIG. 6 is a schematic representation of a bone resorption experiment in which CD14⁺ monocytes were treated with M-CSF and RANKL and one of 4 treatment paradigms for three days (Vehicle, IgG1, rFVIII, or rFVII1Fc), after which monocytes were plated onto bovine cortical bone slices and cultured for an additional 7-10 days, and then stained with toluidine blue to determine bone resorption.

FIGS. 7A-D are visual depictions of bone slices cultured with monocyte-derived osteoclasts previously treated with vehicle (FIG. 7A), IgG1 alone (FIG. 7B), rFVIII (FIG. 7C), or rFVII1Fc (FIG. 7D).

FIG. 8 is a schematic representation of an experiment to determine gene expression in monocyte-derived osteoclasts by treating CD14⁺ monocytes at Day 0 with vehicle, IgG1 alone, rFVIII, or rFVIIIFc, differentiating to monocyte-derived osteoclasts through the addition of M-CSF and RANKL for 7 days, and measuring expression of genes of interest.

FIG. 9 is a graphical representation of gene expression of CD14⁺ monocytes treated with Vehicle (black bars), IgG1 (dark gray bars), rFVIII (light gray bars) or rFVII1Fc (white bars) at Day 0 and differentiated to monocyte-derived osteoclasts at day 7 post-treatment. Markers of differentiation (RANK, NFATC1) and markers of bone resorption activity (CATK, TRAP, MMP9) were measured by quantitative polymerase chain reaction (qPCR) and normalized to an untreated group. ns=not significant; **** p<0.005; n=6-10.

FIG. 10 is a schematic representation of an experiment to determine gene expression and enzymatic activity in monocyte-derived osteoclasts by treating CD14⁺ monocytes at Day 0 with vehicle, IgG1 alone, rFVIII, or rFVII1Fc, differentiating to monocyte-derived osteoclasts through the addition of M-CSF and RANKL for 7 days, and measuring enzymatic activity and expression of genes of interest on day 7.

FIGS. 11A-B are graphical representations of gene expression (FIG. 11A) and enzymatic activity (FIG. 11B) of CD14⁺ monocytes treated with Vehicle (black bars), IgG1 (dark gray bars), rFVIII (light gray bars) or rFVII1Fc (white bars) at Day 0 and differentiated to monocyte-derived osteoclasts at day 7 post-treatment. FIG. 11A depicts antioxidation pathway associated genes (NQO1, GCLC) were measured by qPCR and normalized to the vehicle treated group. FIG. 11B depicts specific NQO1 reductase activity was measured and normalized to the vehicle-treated group. ns=not significant; **** p<0.005; n=10 (FIG. 11A); n=3 (FIG. 11B).

FIG. 12 is a schematic representation of an experiment to determine gene expression and enzymatic activity in osteoclasts by treating CD14⁺ monocytes at Day 0 with vehicle, IgG1 alone, rFVIII, rFVII1Fc, or rFVII1Fc-N297A, differentiating to monocyte-derived osteoclasts through the addition of M-CSF and RANKL for 7 days, and measuring gene expression of osteoclast associated genes.

FIG. 13 is a graphical representation of gene expression of CD14⁺ monocytes treated with Vehicle (black bars), IgG1 (dark gray bars), rFVIII (light gray bars), rFVII1Fc (white bars), or rFVII1Fc-N297A (dashed bars) at Day 0 and differentiated to monocyte-derived osteoclasts at day 7 post-treatment. RANK, NFATC1, CATK, and TRAP levels were measured by qPCR. ns=not significant; **** p<0.005; * p<0.0.05; n=5.

FIGS. 14A-B are a series of density plots displaying immunophenotype as acquired by fluorescence-activated flow cytometry in monocytes treated with rFVIII+IgG1 at different doses and analyzed for surface expression of CD14 and CD51/61. FIG. 14A displays decreasing doses from 75 nM to 7.5 nM of rFVIII+IgG1. FIG. 14B displays decreasing doses from 4.2 nM to 0 nM (vehicle control) of rFVIII+IgG1.

FIGS. 15A-B are a series of density plots displaying immunophenotype as acquired by fluorescence-activated flow cytometry in monocytes treated with rFVII1Fc at different doses and analyzed for surface expression of CD14 and CD51/61. FIG. 15A displays decreasing doses from 75 nM to 7.5 nM of rFVII1Fc. FIG. 15B displays decreasing doses from 4.2 nM to 0 nM (vehicle control) of rFVII1Fc.

FIG. 16 is a graphical representation of the percentage of osteoclast cells compared to vehicle control that were characterized as CD51/61^{high} cells by flow cytometry after treatment with rFVIII+IgG1 (line with circles) or rFVIIIFc (line with squares) at different doses.

FIGS. 17A-D are a series of density plots displaying immunophenotype as acquired by fluorescence-activated flow cytometry in monocytes treated with vehicle (FIG. 17A), rFVII1Fc (FIG. 17B), rFVIII+IgG1 (FIG. 17C), or rFVII1Fc-N297A (FIG. 17D) and analyzed for surface expression of CD16 and CD51/61.

FIGS. 18A-D are a series of density plots displaying immunophenotype as acquired by fluorescence-activated flow cytometry in monocytes treated with rFVII1Fc or rFVIII in the presence of the antigen-binding fragment (Fab) of an FcγR1 blocking antibody (FIGS. 18A-B) or an isotype control Fabnot specifically binding to FcγR1 (FIGS. 18C-D), and analyzed for surface expression of CD16 and CD51/61.

FIGS. 19A-D are a series of density plots displaying immunophenotype as acquired by fluorescence-activated flow cytometry in monocytes treated with rFVII1Fc or rFVIII in the presence of an FcγR2 blocking antibody (FIGS. 19A-B) or an isotype control antibody not specifically binding to FcγR2 (FIGS. 19C-D), and analyzed for surface expression of CD16 and CD51/61.

FIGS. 20A-D are a series of density plots displaying immunophenotype as acquired by fluorescence-activated flow cytometry in monocytes treated with rFVII1Fc or rFVIII in the presence of an FcγR3 blocking antibody (FIGS. 20A-B) or an isotype control antibody not specifically binding to FcγR3 (FIGS. 20C-D), and analyzed for surface expression of CD16 and CD51/61.

FIGS. 21A-D are a series of histograms corresponding to FIG. 17 displaying immunophenotype as acquired by fluorescence-activated flow cytometry in monocytes treated with vehicle (FIG. 21A), rFVII1Fc (FIG. 21B), rFVIII+IgG1 (FIG. 21C), or rFVII1Fc-N297A (FIG. 21D), analyzed for surface expression of CD51/61. The y-axis represents the flow event scaled as a percentage of the maximum count (100%), calculated by the analysis software FlowJo.

FIGS. 22A-D are a series of histograms corresponding to FIG. 18 displaying immunophenotype as acquired by fluorescence-activated flow cytometry in monocytes treated with rFVII1Fc or rFVIII in the presence of the antigen-binding fragment (Fab) of an FcγR1 blocking antibody (FIGS. 22A-B) or an isotype control Fab not specifically binding to FcγR1 (FIGS. 22C-D), and analyzed for surface expression of CD51/61. The y-axis represents the flow event scaled as a percentage of the maximum count (100%), calculated by the analysis software FlowJo.

FIGS. 23A-D are a series of histograms corresponding to FIG. 19 displaying immunophenotype as acquired by fluorescence-activated flow cytometry in monocytes treated with rFVII1Fc or rFVIII in the presence of an FcγR2 blocking antibody (FIGS. 23A-B) or an isotype control antibody not specifically binding to FcγR2 (FIGS. 23C-D), and analyzed for surface expression of CD51/61. The y-axis represents the flow event scaled as a percentage of the maximum count (100%), calculated by the analysis software FlowJo.

FIGS. 24A-D are a series of density plots corresponding to FIG. 20 displaying immunophenotype as acquired by fluorescence-activated flow cytometry in monocytes treated with rFVII1Fc or rFVIII in the presence of an FcγR3 blocking antibody (FIGS. 24A-B) or an isotype control not specifically binding to FcγR3 (FIGS. 24C-D), and analyzed for surface expression of CD51/61. The y-axis represents the flow event scaled as a percentage of the maximum count (100%), calculated by the analysis software FlowJo.

FIGS. 25A-J are visual depictions of monocytes and monocyte-derived osteoclasts in the presence of rFVIII (FIGS. 25A-E) or rFVIIIFc (FIGS. 25F-J) in the presence of an antibody blocking the A2 region of FVIII (GMA8017; FIGS. 25B and 25G), an antibody blocking the A3 region of FVIII (GMA8010; FIGS. 25C and 25H), or in the presence of antibodies blocking the C2 region (GMA8006; FIGS. 25D and 25I; GMA8026; FIGS. 25E and 25J).

FIGS. 26A-E are a series of histograms displaying immunophenotype as acquired by fluorescence-activated flow cytometry in monocytes treated with rFVIII (FIG. 26A) or rFVII1Fc (FIG. 26B) alone or in the presence of von Willebrand Factor (VWF; FIGS. 26C-E) and analyzed for surface expression of CD51/61. The y-axis represents the flow event scaled as a percentage of the maximum count (100%), calculated by the analysis software FlowJo.

### DETAILED DESCRIPTION

The present disclosure is directed to methods used to treat subjects with low bone mineral density (BMD). In an aspect, disclosed herein are methods of treating a subject with hemophilia and low BMD. Certain aspects of the disclosure are directed to methods of treating subjects with hemophilia A and low BMD comprising selecting a subject having hemophilia A and low BMD, and administering to the subject a therapeutically effective amount of a chimeric protein comprising a coagulation factor and an Fc domain. Also disclosed herein are methods for treating subjects with hemophilia A with a chimeric protein wherein administration of the chimeric protein inhibits reduction of BMD in the subject. In certain embodiments, the chimeric protein comprises a FVIII and an Fc region. In certain embodiments, the chimeric protein consists of a FVIII and an Fc region. In various embodiments, the chimeric protein is rFVII1Fc.

### 1. Definitions

Unless defined otherwise, all technical and scientific terms used herein have the same meaning as commonly understood by one of ordinary skill in the art to which this disclosure is related. For example, The Dictionary of Cell and Molecular Biology, 5th ed., 2013, Academic Press; and the Oxford Dictionary of Biochemistry and Molecular Biology, 2d. ed. (rev.), 2006, Oxford University Press, provide one of skill with a general dictionary of many of the terms used in this disclosure.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.” Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used in connection with a numerical value throughout the specification and the claims denotes an interval of accuracy, familiar and acceptable to a person skilled in the art. In general in the Claims, the Summary, and the Detailed Description herein, such interval of accuracy is ±10%. In some embodiments, when used in reference to a particular recited numerical value, “about” means that the value may vary from the recited value by no more than 10%. In some embodiments, when used in reference to a particular recited numerical value, “about” means that the value may vary from the recited value by no more than 1%. For example, as used herein, the expression “about 100” discloses embodiments that include 99 and 101 and all values in between (e.g., 99.1, 99.2, 99.3, 99.4, etc.).

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

As known in the art, “sequence identity” between two polypeptides is determined by comparing the amino acid sequence of one polypeptide to the sequence of a second polypeptide. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full-length of the reference polypeptide sequence and that gaps in homology of up to 5% of the total number of amino acids in the reference sequence are allowed. Other non-limiting examples of algorithms that are suitable for determining percent sequence identity and sequence similarity are the BLAST and BLAST 2.0 algorithms, which are described in Altschul et al, Nucleic Acids Res. 25:3389-3402 (1997) and Altschul et al, J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In certain embodiments, the NCBI BLASTN or BLASTP program is used to align sequences. In certain embodiments, the BLASTN or BLASTP program uses the defaults used by the NCBI. In certain embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In certain embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89: 10915 (1992)); gap costs of existence: 11 and extension: 1; and conditional compositional score matrix adjustment.

### 2. Chimeric Proteins

A “fusion” or “chimeric” polypeptide or protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain with an Ig Fc domain. A fusion protein is created, for example, by chemical synthesis, or by creating and translating a polynucleotide in which the peptide regions are encoded in the desired relationship. A chimeric polypeptide can further comprise a second amino acid sequence associated with the first amino acid sequence by a covalent, non-peptide bond or a non-covalent bond. In certain embodiments, the chimeric protein is a chimeric protein comprising a FVIII protein and an Fc region. For example, the chimeric protein may comprise one FVIII protein fused to one of the polypeptide chains of an Fc dimer. In some embodiments, the chimeric protein comprises one FVIII protein directly fused to the N-terminus of one of the polypeptide chains of an Fc dimer. In some embodiments, the FVIII protein is the only protein that is fused to the Fc dimer. In some embodiments, the chimeric protein comprises one FVIII protein directly fused to the C-terminus of one of the polypeptide chains of an Fc dimer. In some embodiments, the chimeric protein comprising or consisting of a single molecule of recombinant B-domain deleted human FVIII (BDD-rFVIII) fused to one polypeptide chain of the dimeric Fc domain of the human IgG1, with no intervening linker sequence. See, e.g., U.S. Pat. Nos. 9,050,318 and 9,241,978, which are hereby incorporated by reference herein in their entirety. In various embodiments, the chimeric protein is rFVII1Fc. In various embodiments, the rFVII1Fc is the rFVII1Fc referred to as ELOCTA® or ELOCTATE®. rFVII1Fc is disclosed in detail in, e.g., U.S. Patent Application Pub. No. 2018/0360982 A1 and U.S. Pat. Nos. 9,050,318 and 9,241,978, which are hereby incorporated by reference herein in their entireties.

In some embodiments, rFVII1Fc comprises an amino acid sequence according to SEQ ID NO: 1. In some embodiments, rFVII1Fc comprises an amino acid sequence according to amino acids 1-1665 of SEQ ID NO: 1. In some embodiments, rFVII1Fc comprises an amino acid sequence according to SEQ ID NO: 5. In some embodiments, the FVIII portion of the chimeric polypeptide comprises an amino acid sequence at least 95% identical to the amino acid sequence according to SEQ ID NO: 2 and the Fc portion of the chimeric polypeptide comprises an amino acid sequence at least 95% identical to the amino acid sequence according to SEQ ID NO: 5. In some embodiments, FVIII portion of the chimeric polypeptide comprises an amino acid sequence identical to SEQ ID NO: 2 and the Fc portion of the chimeric polypeptide comprises an amino acid sequence identical to SEQ ID NO: 5.

In some embodiments, the chimeric polypeptide comprises a first polypeptide chain and a second polypeptide chain, wherein the first polypeptide chain comprises a FVIII portion and a first Fc portion, and wherein the second polypeptide chain comprises a second Fc portion. In some embodiments, the second polypeptide consists of the second Fc portion. In some embodiments, the first Fc portion has the same amino acid sequence as the second Fc portion. In some embodiments, the first polypeptide chain comprises a FVIII portion and an Fc portion, wherein the FVIII portion is fused to the N-terminus of the Fc portion. In some embodiments, the first polypeptide chain comprises a FVIII portion and an Fc portion, wherein the FVIII portion is fused to the C-terminus of the Fc portion.

In some embodiments, the chimeric polypeptide comprises a first polypeptide chain and a second polypeptide chain, wherein the first polypeptide chain comprises a FVIII portion and a first Fc portion, and wherein the second polypeptide chain comprises a second Fc portion, wherein the first Fc portion and the second Fc portion are associated with each other by a covalent bond. In some embodiments, the first polypeptide chain is covalently bound to the second polypeptide chain via a disulfide bond. In some embodiments, the first polypeptide chain is covalently bound to the second polypeptide chain via two disulfide bonds in a hinge region of the Fc portion.

In some embodiments, the chimeric polypeptide comprises a first polypeptide chain and a second polypeptide chain, wherein the first polypeptide chain comprises a FVIII portion and a first Fc portion, and wherein the second polypeptide chain comprises a second Fc portion, wherein the FVIII portion comprises an amino acid sequence at least 95% identical to the amino acid sequence according to SEQ ID NO: 2 and the Fc portion of the chimeric polypeptide comprises an amino acid sequence at least 95% identical to the amino acid sequence according to SEQ ID NO: 5, and wherein the second Fc portion comprises an amino acid sequence at least 95% identical to the amino acid sequence according to SEQ ID NO: 5.

In some embodiments, the chimeric protein is efmoroctocog alfa.

In some embodiments, the chimeric protein comprises a first polypeptide chain comprising an amino acid sequence at least 95% identical to the amino acid sequence according to SEQ ID NO: 5 and a second polypeptide chain comprising an amino acid sequence at least 95% identical to the amino acid sequence according to SEQ ID NO: 4. In some embodiments, the chimeric protein comprises a first polypeptide chain comprising an amino acid sequence identical to SEQ ID NO: 5 and a second polypeptide chain comprising an amino acid sequence identical to SEQ ID NO: 4. In some embodiments, the chimeric protein does not comprise VWF or a fragment, variant, or mutant thereof.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are normally cleaved from the complete or “full-length” polypeptide to produce a secreted or “mature” form of the polypeptide. In certain embodiments, a native signal peptide or a functional derivative of that sequence retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

In some embodiments, the chimeric protein has been produced by a mammalian cell or mammalian cells. In some embodiments, the chimeric protein has been produced by a human cell or human cells. In some embodiments, the chimeric protein has been produced by human embryonic kidney 293 (HEK293) cells.

“Factor VIII,” abbreviated throughout the instant application as “FVIII,” as used herein, means functional FVIII polypeptide in its normal role in coagulation, unless otherwise specified. Thus, the term FVIII includes variant polypeptides that are functional. A “FVIII protein” is used interchangeably with “FVIII polypeptide” or “FVIII”. Examples of FVIII functions include, but are not limited to, an ability to activate coagulation, an ability to act as a cofactor for factor IX, or an ability to form a tenase complex with factor IX in the presence of Ca²⁺ and phospholipids, which then converts factor X to the activated form Xa. In certain embodiments, the FVIII protein can be a human, non-human primate, porcine, canine, rat, or murine FVIII protein. In certain embodiments, the FVIII protein is a human FVIII protein. In certain embodiments, the FVIII proteins is derived from a human FVIII protein. Non-limiting examples of FVIII proteins that may be derived from human FVIII proteins are disclosed herein and include FVIII proteins with partial or complete deletions of the FVIII B domain, as well as FVIII proteins with mutations in the FVIII B domain such that the FVIII protein is not cleaved by thrombin or has reduced thrombin cleavage compared to a corresponding wild-type FVIII protein. In addition, comparisons between FVIII from humans and other species have identified conserved residues that are likely to be required for function (Cameron et al., Thromb. Haemost. 79:317-22 (1998); U.S. Pat. No. 6,251,632). The full length polypeptide and polynucleotide sequences are known, as are many functional fragments, mutants and modified versions. Various FVIII amino acid and nucleotide sequences are disclosed in, e.g., US Publication Nos. 2015/0158929 A1, 2014/0308280 A1, and 2014/0370035 A1 and International Publication No. WO 2015/106052 A1, each of which is incorporated herein by reference in its entirety. In various embodiments, the FVIII protein is a human FVIII protein, or a functional variant thereof. FVIII polypeptides include, e.g., full-length FVIII, full-length FVIII minus Met at the N-terminus, mature FVIII (minus the signal sequence), mature FVIII with an additional Met at the N-terminus, and/or FVIII with a full or partial deletion of the B domain. FVIII variants include B domain deletions, whether partial or full deletions.

In some embodiments, the FVIII of the chimeric protein or composition of the present disclosure comprises a B domain deleted FVIII. A “B domain” of FVIII, as used herein, is the same as the B domain known in the art that is defined by internal amino acid sequence identity and sites of proteolytic cleavage by thrombin, e.g., residues Ser741-Arg1648 of mature human FVIII. The other human FVIII domains are defined by the following amino acid residues, relative to mature human FVIII: A1, residues Alal-Arg372; A2, residues Ser373-Arg740; A3, residues Ser1690-Ile2032; Cl, residues Arg2033-Asn2172; C2, residues Ser2173-Tyr2332 of mature FVIII. The sequence residue numbers used herein without referring to any SEQ ID Numbers correspond to the FVIII sequence without the signal peptide sequence (19 amino acids) unless otherwise indicated. The A3-C1-C2 sequence, also known as the FVIII heavy chain, includes residues Ser1690-Tyr2332. The remaining sequence, residues Glu1649-Arg1689, is usually referred to as the FVIII light chain activation peptide, or simply the FVIII light chain. The locations of the boundaries for all of the domains, including the B domains, for example for porcine, mouse and canine FVIII are also known in the art. In certain embodiments, the B domain of FVIII is deleted (“B-domain-deleted FVIII” or “BDD FVIII”). An example of a BDD FVIII is REFACTO® (recombinant BDD FVIII).

In some embodiments, a B-domain-deleted FVIII may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, 6,458,563, or Int'l Publ. No. WO 2015106052 A1 (PCT/US2015/010738). In some embodiments, a B-domain-deleted FVIII has a deletion of most of the B domain, but still contains amino-terminal sequences of the B domain that are essential for in vivo proteolytic processing of the primary translation product into two polypeptide chains, as disclosed in WO 91/09122. In some embodiments, a B-domain-deleted FVIII is constructed with a deletion of amino acids 747-1638, i.e., virtually a complete deletion of the B domain. Hoeben R. C., et al. J. Biol. Chem. 265 (13): 7318-7323 (1990). A B-domain-deleted Factor VIII may also contain a deletion of amino acids 771-1666 or amino acids 868-1562 of FVIII. Meulien P., et al. Protein Eng. 2(4): 301-6 (1988). Additional B domain deletions that may be part of certain embodiments include: deletion of amino acids 982 through 1562 or 760 through 1639 (Toole et al., Proc. Natl. Acad. Sci. U.S.A. (1986) 83, 5939-5942), 797 through 1562 (Eaton, et al. Biochemistry (1986) 25:8343-8347), 741 through 1646 (Kaufman (PCT published application No. WO 87/04187)), 747-1560 (Sarver, et al., DNA (1987) 6:553-564), 741 through 1648 (Pasek (PCT application No. 88/00831)), or 816 through 1598 or 741 through 1648 (Lagner (Behring Inst. Mitt. (1988) No 82:16-25, EP 295597)).

In some embodiments, BDD FVIII includes a FVIII polypeptide containing fragments of the B domain that retain one or more N-linked glycosylation sites, e.g., residues 757, 784, 828, 900, 963, or optionally 943, which correspond to the amino acid sequence of the full-length FVIII sequence. Examples of the B-domain fragments include 226 amino acids or 163 amino acids of the B domain as disclosed in Miao, H. Z., et al., Blood 103(a): 3412-3419 (2004), Kasuda, A, et al., J. Thromb. Haemost. 6: 1352-1359 (2008), and Pipe, S. W., et al., J. Thromb. Haemost. 9: 2235-2242 (2011) (i.e., the first 226 amino acids or 163 amino acids of the B domain are retained). In certain embodiments, BDD FVIII further comprises a point mutation at residue 309 (from Phe to Ser) to improve expression of the BDD FVIII protein. See Miao, H. Z., et al., Blood 103(a): 3412-3419 (2004). In various embodiments, the BDD FVIII includes a FVIII polypeptide containing a portion of the B domain, but not containing one or more furin cleavage sites (e.g., Arg1313 and Arg 1648). See Pipe, S. W., et al., J. Thromb. Haemost. 9: 2235-2242 (2011). In some embodiments, the BDD FVIII comprises a single-chain FVIII that contains a deletion in amino acids 765 to 1652 corresponding to the mature full length FVIII (also known as rFVIII-SingleChain and AFSTYLA®). See U.S. Pat. No. 7,041,635. Each of the foregoing deletions may be made in any FVIII sequence.

A great many functional FVIII variants are known in the art. In addition, hundreds of nonfunctional mutations in FVIII have been identified in hemophilia patients, and it has been determined that the effect of these mutations on FVIII function is due more to where they lie within the 3-dimensional structure of FVIII than on the nature of the mutation (Cutler et al., Hum. Mutat. 19:274-8 (2002)), incorporated herein by reference in its entirety. In addition, comparisons between FVIII from humans and other species have identified conserved residues that are likely to be required for function (Cameron et al., Thromb. Haemost. 79:317-22 (1998); U.S. Pat. No. 6,251,632, each incorporated herein by reference in its entirety).

Factor VIII proteins may be present in an active form as either a “processed” FVIII or a “single-chain” FVIII. Such types of processed and single-chain forms are discussed in U.S. Patent Pub. No. 2018/0360982 A1, incorporated herein by reference in its entirety.

In some embodiments, a chimeric polypeptide that has Factor VIII activity comprises a Factor VIII protein and a second portion, wherein the Factor VIII protein is processed Factor VIII comprising two chains, a first chain comprising a heavy chain and a second chain comprising a light chain, wherein said first chain and said second chain are associated by a metal bond. For example, at least about 50%, about 55%, about 60%, about 65%, about 70%, about 75%, about 80%, about 85%, about 90%, about 95%, or about 99% of the chimeric polypeptide comprises a Factor VIII portion that is processed Factor VIII, with the rest of the chimeric polypeptide comprising a Factor VIII portion that is unprocessed (i.e., single-chain FVIII).

In some embodiments, the present disclosure includes a chimeric polypeptide that has Factor VIII activity, wherein the Factor VIII portion is single-chain Factor VIII. In some embodiments, the single-chain Factor VIII can contain an intact intracellular processing site. In some embodiments, at least about 1%, about 5%, about 10%, about 15%, about 20%, about 25%, about 30%, about 35%, or about 40% of the Factor VIII portion of the chimeric polypeptide is single-chain Factor VIII. In another embodiment, at least about 30%, about 40%, about 50%, about 60%, about 70%, about 80%, about 90%, about 95%, or about 99% of the chimeric polypeptide comprises a Factor VIII portion that is single-chain Factor VIII, with the rest of the chimeric polypeptide comprising a Factor VIII portion that is processed Factor VIII. In another aspect, the single-chain FVIII (scFVIII) does not contain an intracellular processing site. For example, the scFVIII comprises a substitution or mutation at an amino acid position corresponding to Arginine 1645, a substitution or mutation at an amino acid position corresponding to Arginine 1648, or a substitution or mutation at amino acid positions corresponding to Arginine 1645 and Arginine 1648 in full-length Factor VIII. In some embodiments, the amino acid substituted at the amino acid position corresponding to Arginine 1645 is a different amino acid from the amino acid substituted at the amino acid position corresponding to Arginine 1648. In certain embodiments, the substitution or mutation is a substitution from arginine to alanine.

In some embodiments, the chimeric polypeptide comprising single-chain Factor VIII has Factor VIII activity at a level comparable to a chimeric polypeptide consisting of two Fc portions and processed Factor VIII, which is fused to one of the two Fc portions, when the Factor VIII activity is measured in vitro by a chromogenic assay. In some embodiments, the chimeric polypeptide comprising single-chain Factor VIII has Factor VIII activity in vivo comparable to a chimeric polypeptide consisting of two Fc portions and processed Factor VIII, which is fused to one of the two Fc portions. In some embodiments, the chimeric polypeptide comprising single-chain Factor VIII has a Factor Xa generation rate comparable to a chimeric polypeptide consisting of two Fc portions and processed Factor VIII, which is fused to one of the two Fc portions. In certain embodiments, single-chain Factor VIII in the chimeric polypeptide is inactivated by activated Protein C at a level comparable to processed Factor VIII in a chimeric polypeptide consisting of two Fc portions and processed Factor VIII. In certain embodiments, the single-chain Factor VIII in the chimeric polypeptide has a Factor IXa interaction rate comparable to processed Factor VIII in a chimeric polypeptide consisting of two Fc portions and processed Factor VIII. In some embodiments, the single-chain Factor VIII in the chimeric polypeptide binds to von Willebrand Factor at a level comparable to processed Factor VIII in a chimeric polypeptide consisting of two Fc portions and the processed Factor VIII.

The present disclosure includes a composition comprising a chimeric polypeptide having Factor VIII activity, wherein at least about 30%, about 40%, about 50%, about 60%, about 70%, about 80%, about 90%, about 95%, about 96%, about 97%, about 98%, about 99%, or about 100% of said polypeptide comprises a Factor VIII portion, which is single-chain Factor VIII, and a second portion, wherein said single-chain Factor VIII is at least 90%, 95%, 99% identical, or is identical to, to amino acid sequence according to SEQ ID NO: 2. In some embodiments, the second portion can be an Fc. In some embodiments, the polypeptide is in the form of a hybrid comprising a second polypeptide, wherein said second polypeptide consists essentially of an Fc. In some embodiments, the polypeptide has a half-life at least one and one-half to six times longer, one and one-half to five times longer, one and one-half to four times longer, one and one-half to three times longer, or one and one-half to two times longer to a polypeptide consisting of the Factor VIII.

### 3. Bone Mineral Density

As used herein, “bone mineral density” or “BMD”, is defined as the bone mineral content measured in a specific bone area. Bone is a dynamic tissue with a relatively high turnover. Bone metabolism is characterized by an equilibrium between bone formation and bone resorption, mediated by osteoblasts and osteoclasts, respectively. The interaction between these bone remodeling cells is mediated by cytokines, growth factors and other proteins.

As used herein, “osteoporosis” refers to a widely recognized disease in which the density and quality of bone are reduced. As used herein, the term “osteoporosis” encompasses all forms of osteoporosis, including both primary osteoporosis and secondary osteoporosis. Osteoporosis is characterized by a severe reduction in BMD, predisposing patients to bone fractures and additional morbidity. Osteoporosis is affected by several factors, most prominently by age, gender, and presence of other diseases. Reactive oxygen species (ROS) also play a role in intracellular signaling during osteoclastogenesis (Domazetovic et al, Clin Cases Miner Bone Metab 2017). Vitamin D deficiency or vitamin D insufficiency has also been associated with low BMD in certain hemophilia populations (Kempton et al, Haemophilia 2015, 21, 568-577). In some embodiments, the osteoporosis is primary osteoporosis. In certain embodiments, the osteoporosis is secondary osteoporosis. In various embodiments, the osteoporosis is associated with hemophilia A. In some embodiments, the osteoporosis is a result of, or is suspected of being a result of, hemophilia A.

Osteoporosis is one of the most common inflammatory bone loss conditions, actively mediated by the immune system (Srivastava R K et al, Front Immunol 2018). The transcriptional factor nuclear factor E2-related factor 2 (NRF2) negatively regulates osteoclastogenesis via antioxidant enzyme upregulation, a mechanism actively inhibited by RANKL (Kanzaki et al, J Biol Chem 2013). Also, the NRF2-regulated enzyme heme oxygenase-1 (HO-1) appears to inhibit osteoclast formation in mice (Florczyk-Soluch et al, Sci Reports 2018).

In certain embodiments, the subject has a vitamin D deficiency. In some embodiments, a vitamin D level of 20 nanograms/milliliter to 50 ng/mL is considered adequate for healthy people. In some embodiments, a vitamin D level less than 12 ng/mL is generally considered to indicate a vitamin D deficiency. In some embodiments, a vitamin D deficiency refers to a vitamin D level less than about 12 ng/mL. In certain embodiments, the subject does not have a vitamin D deficiency. In some embodiments, the vitamin D intake and/or levels of the subject are not considered and/or are unknown. In some embodiments, vitamin D levels in the subject are unknown.

Exemplary biomarkers of bone formation are the bone-specific alkaline phosphatase, procollagen type 1 N-terminal propeptide (P1NP), procollagen type 1 C-terminal propeptide (P1CP) and osteocalcin. Exemplary biomarkers of bone resorption are total alkaline phosphatase in serum, the receptor activator of nuclear factor kappa B (RANKL), osteoprotegerin (OPG), tartrate-resistant acid phosphatase (TRAP), hydroxylysine, hydroxyproline, deoxypyridinoline (DPD), pyridinoline (PYD), bone sialoprotein, cathepsin K, tartrate-resistant acid phosphatase 5b (TRAP5b), matrix metalloproteinase 9 (MMP9), and C- and N-terminal cross-linked telopeptide for type 1 collagen (CTX-1 and NTX-1, respectively). Exemplary biomarkers of bone formation inhibitors are serum levels of Dickkopf-1 (DDK-1) and serum levels of sclerostin (Rodriguez-Merchan and Valentino, Blood Rev 2019; Kuo and Chen, Biomarker Res 2017).

In various embodiments, one or more biomarkers of bone formation, bone resorption, and/or bone loss may be assessed from the peripheral blood of a subject. In various embodiments, one or more biomarkers of bone formation, bone resorption, and/or bone loss may be assessed from the urine of a subject. In various embodiments, one or more biomarkers of bone formation, bone resorption, and/or bone loss may be assessed from a sample of the peripheral blood or urine from a subject.

Assessing biomarker levels from the peripheral blood may be achieved, e.g., using any of several different assays. Non-limiting examples of assays that may be used to determine biomarker levels include High Performance Liquid Chromatography (HPLC), an enzyme-linked immunosorbent assay (ELISA), an enzyme immunoassay, a radioimmunoassay, and a chemiluminescence immunoassay. In some embodiments, chemical analyzers may also be used to determine the levels of biomarker in subject sample, including a standard Technico Auto-analyzer, a Roche COBAS Integra 800, An Olympus AU 5200 analyzer.

In some embodiments, the biomarker is hydroxyproline. In some embodiments, hydroxyproline is assessed from the peripheral blood. In some embodiments, hydroxyproline is assessed from the urine of a subject. In some embodiments, hydroxyproline is assessed from the peripheral blood or urine and is analyzed by the Bergman and Loxley method (Bergman and Loxley, Analytical Chemistry, 1963).

Osteoclasts are large multinucleated cells and are the only cells in the body with bone resorption activity, the ability to break down bone tissue. Osteoclasts are derived from hematopoietic precursors including monocytes, requiring two minimal differentiation factors: RANKL (Receptor Activator of Nuclear Factor KB Ligand) and M-CSF (Macrophage Colony-Stimulating Factor) (Kanzaki H. et al, J Biol Chem 2013). Monocytes are a type of progenitor cell that can differentiate into macrophages, dendritic cells and osteoclasts depending on the stimulatory factors received.

One recent study showed that recombinant FVIII linked to a Fc domain (rFVIIIFc), but not recombinant FVIII alone, skewed human monocyte-derived macrophages to the M2/Mox-like macrophage regulatory phenotype. Kis-Toth et al, Blood Adv., 2(21): 2904-2916 (2018). However, a detailed understanding of the mechanism of loss of BMD in hemophilia is presently unknown.

In certain embodiments, the methods disclosed herein are used to treat subjects having an increased risk of bone fracture. Hemophilia patients are more prone to fractures as compared to healthy individuals. In one study, it was found that severe hemophilia patients are 44% more likely to suffer a bone fracture as compared to moderate and mild hemophilia patients. Gay et al., Br J Haematology. 170:584-593 (2015). In some embodiments, a subject has severe hemophilia. In certain embodiments, a subject has moderate hemophilia. In various embodiments, a subject has mild hemophilia.

As used herein, the term “fracture risk” is defined as an increase in the likelihood of bone fracture based on known risk factors. Fracture risk based on known risk factors may be determined by a clinician and/or by standardized tools such as the FRAX fracture risk assessment tool. BMD may be considered a risk factor for fracture risk. Generally, as BMD decreases, risk of fracture increases.

As used herein, FRAX refers to the fracture risk assessment tool developed at the University of Sheffield. See generally Kanis, J. A., et al. Osteoporosis Intl. 21.2: 407-413 (2010). FRAX calculates 10-year probability of hip or osteoporotic fracture. FRAX calculates fracture risk based on age, sex, weight, height, history of fracture, family history of fractured hip, smoking status, use of glucocorticoids, presence or absence of rheumatoid arthritis, secondary osteoporosis, alcohol intake and bone mineral density. A one-year risk fracture is equal to 10% of the output of a ten year risk fracture (i.e., a ten year risk fracture of 60% would equate to a one year risk fracture of 6%).

In certain embodiments, the BMD of a hemophilia patient is determined following a specific event, including a bleeding event or a bone fracture. BMD can be tested, for example, by Dual X-ray Absorptiometry (DXA) or Dual-Energy X-ray Absorptiometry (DEXA). BMD may be measured as grams per centimeter squared (g/cm²). To analyze BMD across a population, BMD may be compared to an average “T-Score” for healthy young adults. This T-Score is the difference in mean BMD between a patient and a group of healthy average young adults of the same sex, measured in standard deviation (SD). For example, a T-Score of −1.0 or higher (less negative) may be considered normal. A T-score below −1.0 (more negative) may be indicative of osteopenia. A T-Score below −2.5 may be considered indicative of osteoporosis. A BMD test may measure bone mineral density at the hip or lumbar spine. A BMD test may also measure bone mineral density at the lower arm, wrist, finger or heel. BMD may also be compared to an average “Z-score”. This Z-score is the difference in mean BMD between a patient and a group of healthy, age- and sex-matched controls, measured in standard deviation. A Z-score may be useful for the diagnosis of secondary osteoporosis. A Z-score below −2.0 may be indicative of low bone mineral density. For additional details regarding bone densitometry, including T-scores and Z-scores, see Cummings et al., JAMA 288(15):1889-1897 (2002), the entire content of which is incorporated herein by reference.

In certain embodiments, the T-score is used to assess BMD in subjects who are at least 20 years of age. In certain embodiments, the T-score is used to assess BMD in subjects who are at least 30 years of age. In certain embodiments, the T-score is used to assess BMD in subjects who are at least 40 years of age. In certain embodiments, the T-score is used to assess BMD in subjects who are at least 50 years of age.

In certain embodiments, the Z-score is used to assess BMD in subjects who are less than 30 years of age. In certain embodiments, the Z-score is used to assess BMD in subjects who are less than 20 years of age.

In some embodiments, a subject with hemophilia A and low BMD has bone density that is between 1 and 2.5 standard deviations below the young adult mean. In some embodiments, a subject with hemophilia A and low BMD has bone density that is 2.5 standard deviations or more below the young adult mean. In some embodiments, the subject has bone density that is less than the average bone density for a subject of the same age and gender. In some embodiments, the subject has bone density that is at least 5%, 6%, 7%, 8%, 9%, or 10% less than the average bone density for a subject of the same age and gender. In some embodiments, the subject has bone density that is at least 10% less than the average bone density for a subject of the same age and gender. In some embodiments, the BMD is measured at the lumbar spine. In some embodiments, the BMD is measured at the hip. In some embodiments, the BMD is measured at an arm. In some embodiments, the BMD is measured at a leg. In some embodiments, the BMD is measured at a knee. In some embodiments, the BMD is measured at a wrist. In some embodiments, the BMD is measured at a finger. In some embodiments, the BMD is measured at a heel. In some embodiments, a subject who has low BMD has 10% or 15% lower BMD at a particular site compared to a corresponding subject (or population of corresponding subjects) that does not have hemophilia A.

In some embodiments, a subject can be identified as having low BMD using risk factors. Risk factors for low BMD include age, gender, ethnicity, hemophilic arthropathy, reduced physical activity, chronical viral infection (e.g. HIV or HCV), vitamin D deficiency, low body mass index (BMI), and/or hypogonadism. See Kempton C L et al. Haemophilia 21(5):568-77 (2015). Other risk factors can be evaluated according to current accepted clinical guidelines and practices as known in the art.

If the subject is determined to have low BMD, the methods disclosed herein can be used to inhibit the reduction of BMD in the subject and/or protect against further reduction in BMD in the subject. If a subject is currently being treated with another FVIII replacement therapy or another hemophilia A therapy, a change in treatment plan to the methods disclosed herein may be considered in order to inhibit the reduction of BMD in the subject and/or protect against further reduction in BMD in the subject over time.

As detailed in the Examples disclosed herein, administration of rFVII1Fc to human macrophages treatment effectively inhibited monocyte-derived osteoclast formation and function in vitro. This finding suggests that replacement therapy with rFVII1Fc may have potential immunoregulatory benefits on bone health in hemophilia A patients. While the precise mechanism remains unknown, and without being bound by any scientific theory, rFVII1Fc may protect against reduction in BMD in hemophilia A patients by promoting the immune milieu in hemophiliacs toward an antioxidant, tolerogenic, and less osteoporotic state.

### 4. Hemophilia

The three main forms of hemophilia are hemophilia A (Factor VIII deficiency), hemophilia B (Factor IX deficiency or “Christmas disease”) and hemophilia C (Factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or Factor XIII, Bernard-Soulier syndrome, which is a defect or deficiency in GPIb. GPIb, the receptor for von Willebrand Factor (VWF), can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk. As used herein, hemophilia may be graded by category. For instance, it may be classified as “mild”, “moderate” or “severe”. Hemophilia A has three grades of severity defined by FVIII plasma levels of 1% (compared to normal) or less (“severe”), 2% to 5% (“moderate”), and 6 to 30% (“mild”). White et al. Thromb. Haemost. 85:560 (2001).

“Treat”, “treatment”, “treating”, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one aspect, the methods disclosed herein are methods of treating a subject with hemophilia A. In certain embodiments, treating comprises reducing or preventing the likelihood of a bleeding episode in a subject and also improving BMD or slowing reduction of BMD in a subject, e.g., compared to a corresponding subject who is treated with rFVIII replacement. In certain embodiments, treating comprises reducing the risk of a bleeding episode in a subject and also reducing the risk of a bone fracture in a subject, e.g., compared to a corresponding subject who is treated with rFVIII replacement. In certain embodiments, treating comprises reducing the severity of a bleeding episode in a subject and also improving BMD or slowing reduction of BMD in a subject, e.g., compared to a corresponding subject who is treated with rFVIII replacement. In certain embodiments, treating comprises reducing the severity of bleeding episode in a subject and also reducing the risk of a bone fracture in a subject, e.g., compared to a corresponding subject who is treated with rFVIII replacement. In some embodiments, treatment comprises prophylactic treatment. In some embodiments, treatment comprises on-demand treatment.

Several treatment options for hemophilia A are currently available, including conventional FVIII replacement (e.g. ADVATE®/octocog alfa, AFSTYLA®/lonoctocog alfa NUWIQ®/simoctocog alfa) and extended half-life FVIII replacement therapies (e.g. ELOCTATE®/efmoroctocog alfa, ESPEROCT®/turoctocog alfa pegol, and ADYNOVATE®/rurioctocog alfa pegol). Other non-replacement therapies are now available as well, such as emicizumab. For a review, see Peters & Harris, Nat Rev Drug Disc. (2018); Weyand & Pipe, Blood, 133(5): 389-398 (2019). The impact of treatments such as octocog alfa, lonoctocog alfa, simoctocog alfa, turoctocog alfa, and rurioctocog alfa pegol on BMD and osteoporosis are unknown.

Data provided herein have demonstrated that treatment using rFVII1Fc may provide additional osteoprotective benefits to hemophilia A patients by inhibiting BMD loss over time. These bone health benefits were not observed using treatment with rFVIII alone, suggesting that these benefits are unique to rFVII1Fc, most likely due to the presence of the Fc domain on the chimeric protein. As such, rFVII1Fc may be a superior choice of treatment for hemophilia A subjects who have low BMD, osteoporosis, and/or increased fracture risk. Furthermore, since BMD reduction is a progressive disease and begins at a young age in subjects with hemophilia A, rFVII1Fc may be a superior choice of treatment for any hemophilia A subject at risk for developing or having low BMD.

In various embodiments, a subject with hemophilia A has adequate clotting with a treatment other than rFVII1Fc, but has low BMD, osteoporosis, and/or increased fracture risk. In some embodiments, a subject with hemophilia A has adequate clotting with a fusion protein comprising rFVIII and a half-life extending moiety (such as albumin or polyethylene glycol), but has low BMD, osteoporosis, and/or increased fracture risk. In some embodiments, a subject with hemophilia A has adequate clotting with rFVIII, but has low BMD, osteoporosis, and/or increased fracture risk. In certain embodiments, a subject with hemophilia A has adequate clotting with a pro-clotting bispecific antibody (e.g., a bispecific antibody that binds Factor IX and Factor X such as emicizumab or emicizumab-kxwh), but has low BMD, osteoporosis, and/or increased fracture risk. In some embodiments, the subject has osteopenia. In some embodiments, the subject has osteoporosis. In some embodiments, the subject has increased fracture risk.

In various embodiments, adequate clotting in a subject with hemophilia A is a FVIII activity of at least 1%, 2%, 3%, 4%, or at least 5% between doses. For example, in some embodiments the FVIII activity between doses does not drop to less than 1%, 2%, 3%, 4%, or 5% between doses. In certain embodiments, FVIII activity is measured with an activated partial thromboplastin time (aPTT) assay. In various embodiments, adequate clotting in a subject with hemophilia A is an annualized bleeding rate (ABR) of equal to or less than 5 bleeds. In various embodiments, adequate clotting in a subject with hemophilia A is an ABR of equal to or less than 4 bleeds. In various embodiments, adequate clotting in a subject with hemophilia A is an ABR of equal to or less than 3 bleeds. In various embodiments, adequate clotting in a subject with hemophilia A is an ABR of equal to or less than 2 bleeds. In various embodiments, adequate clotting in a subject with hemophilia A is an ABR of equal to or less than 1 bleed. In certain embodiments, FVIII activity is measured with a chromogenic assay. In various embodiments, adequate clotting in a subject with hemophilia A is an annualized bleeding rate (ABR) of less than 5 bleeds. In various embodiments, adequate clotting in a subject with hemophilia A is an ABR of less than 4 bleeds. In various embodiments, adequate clotting in a subject with hemophilia A is an ABR of less than 3 bleeds. In various embodiments, adequate clotting in a subject with hemophilia A is an ABR of less than 2 bleeds. In various embodiments, adequate clotting in a subject with hemophilia A is an ABR of less than 1 bleed.

As used herein the term “prophylactic treatment” refers to the administration of a therapy for the treatment of hemophilia, where such treatment is intended to prevent or reduce the severity of one or more symptoms of hemophilia, e.g., bleeding episodes, e.g., one or more spontaneous bleeding episodes, and/or joint damage. See Jimenez-Yuste et al., Blood Transfus. 12(3):314-19 (2014). To prevent or reduce the severity of such symptoms, e.g., bleeding episodes and the progression of joint disease, hemophilia A patients may receive regular infusions of clotting factor as part of a prophylactic treatment regimen. The basis of such prophylactic treatment is the observation that hemophilia patients with a clotting factor level, e.g., a FVIII level, of 1% or more rarely experience spontaneous bleeding episodes and have fewer hemophilia-related comorbidities as compared to patients with severe hemophilia. See, e.g., Coppola A. et al, Semin. Thromb. Hemost. 38(1): 79-94 (2012). Health care practitioners treating these hemophilia patients surmised that maintaining factor levels at around 1% with regular infusions could potentially reduce the risk of hemophilia symptoms, including bleeding episodes and joint damage. See id. Subsequent research has confirmed these benefits in pediatric hemophilia patients receiving prophylactic treatment with clotting factor, rendering prophylactic treatment the goal for people with severe hemophilia. See id.

A “prophylactic” treatment can also refer to the preemptive administration of the composition described herein, e.g., a chimeric polypeptide, to a subject in order to control, manage, prevent, or reduce the occurrence or severity of one or more symptoms of hemophilia A, e.g., bleeding episodes. Prophylactic treatment with a clotting factor, e.g., FVIII, is the standard of care for subjects with severe hemophilia A. See, e.g., Oldenburg, Blood 125:2038-44 (2015). In some embodiments, prophylactic treatment refers to administering a composition disclosed herein to a subject in need thereof to reduce the occurrence of one or more symptom of hemophilia A. A prophylactic treatment can include administration of multiple doses. The multiple doses used in prophylactic treatment are typically administered at particular dosing intervals. In certain embodiments, the annualized bleeding rate can be reduced to less than or equal to 10, less than or equal to 9, less than or equal to 8, less than or equal to 7, less than or equal to 6, less than or equal to 5, less than or equal to 4, less than or equal to 3, less than or equal to 2, or less than or equal to 1. In certain embodiments, the annualized bleeding rate can be reduced to less than 10, less than 9, less than 8, less than 7, less than 6, less than 5, less than 4, less than 3, less than 2, or less than 1.

The term “on-demand treatment” or “episodic treatment” refers to the “as needed” administration of a chimeric molecule in response to symptoms of hemophilia A, e.g., a bleeding episode, or before an activity that can cause bleeding. In an aspect, the on-demand treatment can be given to a subject when bleeding starts, such as after an injury, or when bleeding is expected, such as before surgery. In an aspect, the on-demand treatment can be given prior to activities that increase the risk of bleeding, such as contact sports. In some embodiments, the on-demand treatment is given as a single dose. In some embodiments, the on-demand treatment is given as a first dose, followed by one or more additional doses. When the chimeric polypeptide is administered on-demand, the one or more additional doses can be administered at least about 12 hours, at least about 24 hours, at least about 36 hours, at least about 48 hours, at least about 60 hours, at least about 72 hours, at least about 84 hours, at least about 96 hours, at least about 108 hours, or at least about 120 hours after the first dose. It should be noted, however, that the dosing interval associated with on-demand treatment is not the same as the dosing interval used for prophylactic treatment.

As used herein, the term “dose” refers to a single administration of a composition to a subject. A single dose can be administered all at once, e.g., as a bullous, or over a period of time, e.g., via an intravenous infusion. The term “multiple doses” means more than one dose, e.g., more than one administration. When referring to co-administration of more than one composition, a dose of composition A can be administered concurrently with a dose of composition B. Alternatively, a dose of composition A can be administered before or after a dose of composition B. In some embodiments, composition A and composition B are combined into a single formulation.

In certain embodiments, “dose” refers to a therapeutically effective amount of a chimeric protein. In certain embodiments, the dose refers to a therapeutically effective amount of rFVII1Fc. In certain embodiments, a therapeutically effective amount of rFVII1Fc is from about 10 IU/Kg to about 300 IU/kg. In some embodiments, a therapeutically effective amount of rFVII1Fc is from about 20 IU/Kg to about 300 IU/kg. In some embodiments, a therapeutically effective amount of rFVII1Fc is about 20 IU/kg to about 250 IU/kg, about 20 IU/kg to about 200 IU/kg, about 20 IU/kg to about 190 IU/kg, about 20 IU/kg to about 180 IU/kg, about 20 IU/kg to about 170 IU/kg, about 20 IU/kg to about 160 IU/kg, about 20 IU/kg to about 150 IU/kg, about 20 IU/kg to about 140 IU/kg, about 20 IU/kg to about 130 IU/kg, from about 20 IU/kg to about 120 IU/kg, from about 20 IU/kg to about 110 IU/kg, from about 20 IU/kg to about 100 IU/kg, from about 20 IU/kg to about 90 IU/kg, from about 20 IU/kg to about 80 IU/kg, from about 20 IU/kg to about 70 IU/kg, from about 20 IU/kg to about 60 IU/kg, from about 25 IU/kg to about 100 IU/kg, from about 25 IU/kg to about 90 IU/kg, from about 25 IU/kg to about 80 IU/kg, from about 25 IU/kg to about 70 IU/kg, from about 25 IU/kg to about 65 IU/kg. In an embodiment, a therapeutically effective amount of rFVII1Fc is from about 20 IU/kg to about 100 IU/kg. In some embodiments, a therapeutically effective amount of rFVII1Fc is from about 25 IU/kg to about 65 IU/kg. In some embodiments, a therapeutically effective amount of rFVII1Fc is from about 20 IU/kg to about 100 IU/kg, from about 30 IU/kg to about 100 IU/kg, from about 40 IU/kg to about 100 IU/kg, from about 50 IU/kg to about 100 IU/kg, from about 60 IU/kg to about 100 IU/kg, from about 70 IU/kg to about 100 IU/kg, from about 80 IU/kg to about 100 IU/kg, from about 90 IU/kg to about 100 IU/kg, from about 20 IU/kg to about 90 IU/kg, from about 20 IU/kg to about 80 IU/kg, from about 20 IU/kg to about 70 IU/kg, from about 20 IU/kg to about 60 IU/kg, from about 20 IU/kg to about 50 IU/kg, from about 20 IU/kg to about 40 IU/kg, or from about 20 IU/kg to about 30 IU/kg.

In other embodiments, a therapeutically effective amount of rFVII1Fc is about 10 IU/kg, about 15 IU/kg, about 20 IU/kg, about 25 IU/kg, about 30 IU/kg, about 35 IU/kg, about 40 IU/kg, about 45 IU/kg, about 50 IU/kg, about 55 IU/kg, about 60 IU/kg, about 65 IU/kg, about 70 IU/kg, about 75 IU/kg, about 80 IU/kg, about 85 IU/kg, about 90 IU/kg, about 95 IU/kg, about 100 IU/kg, about 105 IU/kg, about 110 IU/kg, about 115 IU/kg, about 120 IU/kg, about 125 IU/kg, about 130 IU/kg, about 135 IU/kg, about 140 IU/kg, about 145 IU/kg, about 150 IU/kg, about 155 IU/kg, about 160 IU/kg, about 165 IU/kg, about 170 IU/kg, about 175 IU/kg, about 180 IU/kg, about 185 IU/kg, about 190 IU/kg, about 195 IU/kg, about 200 IU/kg, about 225 IU/kg, about 250 IU/kg, about 275 IU/kg, or about 300 IU/kg. In an embodiment, a therapeutically effective amount of rFVII1Fc is about 50 IU/kg. In another embodiment, a therapeutically effective amount of rFVII1Fc is about 100 IU/kg. In another embodiment, a therapeutically effective amount of rFVII1Fc is about 200 IU/kg.

As used herein, the term “interval” or “dosing interval” refers to the amount of time that elapses between a first dose of composition A and a subsequent dose of the same composition administered to a subject. A dosing interval can refer to the time that elapses between a first dose and a second dose, or a dosing interval can refer to the amount of time that elapses between multiple doses.

The term “dosing frequency” as used herein refers to the number of doses administered per a specific dosing interval. For example, a dosing frequency can be written as once a week, once every two weeks, etc. Therefore, a dosing interval of 7 days can be also written as a dosing interval of once in 7 days or once every week, or once a week.

In some embodiments, the chimeric protein is rFVII1Fc and is administered to the subject at a dosing interval of about two days, about three days, about four days, about five days, about six days, about seven days, about eight days, about nine days, about ten days, about 11 days, about 12 days, about 13 days, about 14 days, about 15 days, about 16 days, about 17 days, about 18 days, about 19 days, about 20 days, about 21 days, about 22 days, about 23 days, or about 24 days. In some embodiments, rFVII1Fc is administered to the human at a dosing interval of about 25 days, about 26 days, about 27 days, about 28 days, about 29 days, about 30 days, about 45 days, or about 60 days.

In some embodiments, rFVII1Fc is administered at a dosing interval of about 1 to about 14 days, about 1 to about 13 days, about 1 to about 12 days, about 1 to about 11 days, about 1 to about 10 days, about 1 to about 9 days, about 1 to about 8 days, about 1 to about 7 days, about 1 to about 6 days, about 1 to about 5 days, about 1 to about 4 days, about 1 to about 3 days, about 1 to about 2 days, about 2 to about 14 days, about 3 to about 14 days, about 4 to about 14 days, about 5 to about 14 days, about 6 to about 14 days, about 7 to about 14 days, about 8 to about 14 days, about 9 to about 14 days, about 10 to about 14 days, about 11 to about 14 days, about 12 to about 14 days, about 13 to about 14 days, or about 5 to about 10 days. In other embodiments, rFVII1Fc is administered at a dosing interval of about 1 to about 21 days, about 1 to about 20 days, about 1 to about 19 days, about 1 to about 18 days, about 1 to about 17 days, about 1 to about 16 days, about 1 to about 15 days, about 1 to about 14 days, about 1 to about 13 days, about 1 to about 12 days, about 1 to about 11 days, about 1 to about 10 days, about 1 to about 9 days, about 1 to about 8 days, about 1 to about 7 days, about 1 to about 6 days, about 1 to about 5 days, about 1 to about 4 days, about 1 to about 3 days, about 1 to about 2 days, about 2 to about 21 days, about 3 to about 21 days, about 4 to about 21 days, about 5 to about 21 days, about 6 to about 21 days, about 7 to about 21 days, about 8 to about 21 days, about 9 to about 21 days, about 10 to about 21 days, about 11 to about 21 days, about 12 to about 21 days, about 13 to about 21 days, about 14 to about 21 days, about 15 to about 21 days, about 16 to about 21 days, about 17 to about 21 days, about 18 to about 21 days, about 19 to about 21 days, about 20 to about 21 days, about 5 to about 10 days, about 10 to about 15 days, about 15 to about 20 days. In some embodiments, rFVII1Fc is administered at a dosing interval of about 2 to about 6 days. In some embodiments, rFVII1Fc is administered at a dosing interval of about 3 to about 5 days.

In various embodiments, the therapeutically effective amount of rFVII1Fc is 25-65 IU/kg (25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 62, 64, or 65 IU/kg) and the dosing interval is once every 3-5, 3-6, 3-7, 3, 4, 5, 6, 7, or 8 or more days, or three times per week, or no more than three times per week. In some embodiments, the therapeutically effective amount of rFVII1Fc is 65 IU/kg and the dosing interval is once weekly, or once every 6-7 days. The doses can be administered repeatedly as long as they are necessary (e.g., at least 10, 20, 28, 30, 40, 50, 52, or 57 weeks, at least 1, 2, 3, 4, 5, 6, 7, 8, 9, or 10 years). In various embodiments, the therapeutically effective amount of rFVII1Fc is about 25-65 IU/kg and the dosing interval is once every 3-5 days.

### 5. Methods

Methods

An aspect of the present disclosure is a method of treating a subject with hemophilia and low BMD. The method comprises selecting a subject having hemophilia A and low BMD, and administering to the subject a therapeutically effective amount of a chimeric protein comprising a recombinant FVIII protein and a Fc domain (rFVII1Fc), wherein administration of the chimeric protein inhibits reduction of BMD in the subject. In some embodiments, the Fc domain is the IgG1. In some embodiments, the Fc domain is the Fc domain of human IgG1. In some embodiments, the chimeric protein is rFVII1Fc.

Similarly, an aspect of the present disclosure is a chimeric protein comprising a recombinant FVIII protein and a Fc domain for use in treating a subject with hemophilia A and low BMD.

In certain embodiments, the chimeric protein comprises an amino acid sequence at least 95% identical to an amino acid sequence according to SEQ ID NO: 1. In certain embodiments, the chimeric protein comprises an amino acid sequence at least 99% identical to an amino acid sequence according to SEQ ID NO: 1. In certain embodiments, the chimeric protein comprises an amino acid sequence 100% identical to SEQ ID NO: 1.

In certain embodiments, the chimeric protein comprises an amino acid sequence at least 95% identical to an amino acid sequence according to SEQ ID NO: 2. In certain embodiments, the chimeric protein comprises an amino acid sequence at least 99% identical to an amino acid sequence according to SEQ ID NO: 2. In certain embodiments, the chimeric protein comprises an amino acid sequence 100% identical to SEQ ID NO: 2.

In certain embodiments, the chimeric protein comprises an amino acid sequence 100% identical to SEQ ID NO: 5.

In certain embodiments, the chimeric protein is administered at a dose of 25-65 IU/kg every 3-5 days.

In certain embodiments, BMD in the subject is measured by Dual X-Ray Absorptiometry (DXA).

In certain embodiments, the subject is 50 years of age or older.

In certain embodiments, the subject is younger than 50 years of age.

In certain embodiments, BMD in the subject is determined by T-score. In certain embodiments, BMD in the subject is determined by T-score. In certain embodiments, the subject is 50 years of age or older, and BMD in the subject is determined by T-score.

In certain embodiments, the subject is determined to have low BMD if the subject has a T-score of less than −1.0. In certain embodiments, the subject is determined to have low BMD and osteopenia if the subject has T-score between −1.0 and −2.4. In certain embodiments, the subject is determined to have low BMD and osteoporosis if the subject has a T-score of less than −2.5.

In certain embodiments, BMD in the subject is determined by Z-score. In certain embodiments, the subject is less than 50 years of age, and BMD in the subject is determined by Z-score.

In certain embodiments, the subject is determined to have low BMD if the subject has a Z-score of less than −2.0.

In certain embodiments, the subject is predicted to have low BMD based on levels of one or more biomarkers of bone formation, bone resorption, and/or bone loss.

In certain embodiments, the biomarker is assessed from the peripheral blood or urine of the subject.

In certain embodiments, the one or more biomarkers of bone formation is selected from the group consisting of bone-specific alkaline phosphatase, procollagen type 1 N-terminal propeptide (P1NP), procollagen type 1 C-terminal propeptide (P1CP), osteocalcin, and any combination thereof.

In certain embodiments, the one or more biomarkers of bone resorption is selected from the group consisting of total alkaline phosphatase in serum, the receptor activator of nuclear factor kappa B (RANKL), osteoprotegerin (OPG), tartrate-resistant acid phosphatase (TRAP), hydroxylysine, hydroxyproline, deoxypyridinoline (DPD), pyridinoline (PYD), bone sialoprotein, cathepsin K, tartrate-resistant acid phosphatase 5b (TRAP5b), matrix metalloproteinase 9 (MMP9), C-terminal cross-linked telopeptide for type 1 collagen (CTX-1), N-terminal cross-linked telopeptide for type 1 collagen (NTX-1), and any combination thereof.

An aspect of the present disclosure is a method of treating a subject with hemophilia A and an increased risk of bone fracture. The method comprises: (i) selecting a subject having hemophilia A and an increased risk of fracture, and (ii) administering to the subject a therapeutically effective amount of a chimeric protein comprising a recombinant FVIII protein and a Fc domain, wherein administration of the chimeric protein reduces the risk of fracture in the subject.

In certain embodiments, the chimeric protein comprises an amino acid sequence at least 95% identical to an amino acid sequence according to SEQ ID NO: 1. In certain embodiments, the chimeric protein comprises an amino acid sequence at least 99% identical to an amino acid sequence according to SEQ ID NO: 1. In certain embodiments, the chimeric protein comprises an amino acid sequence 100% identical to SEQ ID NO: 1.

In certain embodiments, the chimeric protein comprises an amino acid sequence at least 95% identical to an amino acid sequence according to SEQ ID NO: 2. In certain embodiments, the chimeric protein comprises an amino acid sequence at least 99% identical to an amino acid sequence according to SEQ ID NO: 2. In certain embodiments, the chimeric protein comprises an amino acid sequence 100% identical to SEQ ID NO: 2.

In certain embodiments, the chimeric protein comprises an amino acid sequence at least 95% identical to an amino acid sequence according to SEQ ID NO: 5. In certain embodiments, the chimeric protein comprises an amino acid sequence at least 99% identical to an amino acid sequence according to SEQ ID NO: 5. In certain embodiments, the chimeric protein comprises an amino acid sequence 100% identical to SEQ ID NO: 5.

In certain embodiments, the chimeric protein is administered at a dose of 25-65 IU/kg every 3-5 days.

In certain embodiments, the risk of fracture in the subject is determined by the fracture risk assessment tool (FRAX).

In certain embodiments, the risk of fracture in the subject is determined by assessment of low BMD risk factors. In certain embodiments, the low BMD risk factors are selected from the group consisting of arthropathy, reduced physical activity, infection with HIV or HCV, vitamin D deficiency, low body mass index (BMI), hypogonadism, and any combination thereof.

An aspect of the present disclosure is a method of reducing the rate of bone mineral density (BMD) loss in a subject. The method comprises: (i) selecting a subject with low BMD; and (ii) administering to the subject a therapeutically effective amount of a chimeric protein comprising a coagulation factor and a Fc domain, such that administration of the chimeric protein reduces the rate of BMD loss in the subject.

In certain embodiments, the chimeric protein comprises an amino acid sequence at least 95% identical to an amino acid sequence according to SEQ ID NO: 1. In certain embodiments, the chimeric protein comprises an amino acid sequence at least 99% identical to an amino acid sequence according to SEQ ID NO: 1. In certain embodiments, the chimeric protein comprises an amino acid sequence 100% identical to SEQ ID NO: 1.

In certain embodiments, the chimeric protein comprises an amino acid sequence at least 95% identical to an amino acid sequence according to SEQ ID NO: 2. In certain embodiments, the chimeric protein comprises an amino acid sequence at least 99% identical to an amino acid sequence according to SEQ ID NO: 2. In certain embodiments, the chimeric protein comprises an amino acid sequence 100% identical to SEQ ID NO: 2.

In certain embodiments, the chimeric protein comprises an amino acid sequence at least 95% identical to an amino acid sequence according to SEQ ID NO: 5. In certain embodiments, the chimeric protein comprises an amino acid sequence at least 99% identical to an amino acid sequence according to SEQ ID NO: 5. In certain embodiments, the chimeric protein comprises an amino acid sequence 100% identical to SEQ ID NO: 5.

In certain embodiments, the chimeric protein is administered at a dose of 25-65 IU/kg every 3-5 days.

An aspect of the present disclosure is a method of treating a subject with hemophilia A and a fracture. The method comprises selecting a subject having hemophilia and a fracture, and administering to the subject a therapeutically effective amount of a chimeric protein comprising a recombinant FVIII protein and a Fc domain.

In certain embodiments, the chimeric protein comprises an amino acid sequence at least 95% identical to an amino acid sequence according to SEQ ID NO: 1. In certain embodiments, the chimeric protein comprises an amino acid sequence at least 99% identical to an amino acid sequence according to SEQ ID NO: 1. In certain embodiments, the chimeric protein comprises an amino acid sequence 100% identical to SEQ ID NO: 1.

In certain embodiments, the chimeric protein comprises an amino acid sequence at least 95% identical to an amino acid sequence according to SEQ ID NO: 2. In certain embodiments, the chimeric protein comprises an amino acid sequence at least 99% identical to an amino acid sequence according to SEQ ID NO: 2. In certain embodiments, the chimeric protein comprises an amino acid sequence 100% identical to SEQ ID NO: 2.

In certain embodiments, the chimeric protein comprises an amino acid sequence at least 95% identical to an amino acid sequence according to SEQ ID NO: 5. In certain embodiments, the chimeric protein comprises an amino acid sequence at least 99% identical to an amino acid sequence according to SEQ ID NO: 5. In certain embodiments, the chimeric protein comprises an amino acid sequence 100% identical to SEQ ID NO: 5.

In accordance with each of the foregoing aspects and embodiments of the present disclosure, in some embodiments the subject has mild hemophilia A.

In accordance with each of the foregoing aspects and embodiments of the present disclosure, in some embodiments the subject has moderate hemophilia A.

In accordance with each of the foregoing aspects and embodiments of the present disclosure, in some embodiments the subject has severe hemophilia A.

In accordance with each of the foregoing aspects and embodiments of the present disclosure, in some embodiments the subject is human.

Certain aspects of the present disclosure are directed to a method of increasing bone mineral density (BMD) and prophylactically treating bleeding episodes in a subject who has hemophilia A, the method comprising: (i) identifying a subject who is receiving treatment for hemophilia A with a FVIII protein without an Fc portion, wherein the subject has had adequate blood clotting during the treatment, and wherein the subject has low BMD; and (ii) discontinuing treatment with the FVIII protein without an Fc portion and administering to the subject a therapeutically effective amount of a chimeric protein comprising a recombinant FVIII protein and a Fc domain (rFVII1Fc), wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject who has hemophilia A, the method comprising: (i) identifying a subject who is receiving treatment for hemophilia A with a non-factor replacement protein, wherein the subject has had adequate blood clotting during the treatment, and wherein the subject has low BMD; and (ii) discontinuing treatment with the non-factor replacement protein and administering to the subject a therapeutically effective amount of a chimeric protein comprising a recombinant FVIII protein and a Fc domain (rFVII1Fc), wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and low BMD, and wherein administration of the chimeric protein increases BMD and prophylactically treats bleeding episodes in the subject.

Certain aspects of the present disclosure are directed to a method of reducing the risk of fracture and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and an increased risk of fracture, and wherein administration of the chimeric protein reduces the risk of fracture and prophylactically treats bleeding episodes in the subject.

Certain aspects of the present disclosure are directed to a method of reducing the rate of bone mineral density (BMD) loss and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and BMD loss, and wherein administration of the chimeric protein reduces the rate of BMD loss and prophylactically treats bleeding episodes in the subject.

Certain aspects of the present disclosure are directed to a method of increasing bone mineral density (BMD) and prophylactically treating bleeding episodes in a subject who has hemophilia A and is being treated with a FVIII protein without an Fc portion, the method comprising discontinuing treatment with the FVIII protein without an Fc portion and administering to the subject a therapeutically effective amount of a chimeric protein comprising a recombinant FVIII protein and a Fc domain (rFVII1Fc), wherein the subject has been identified as having low BMD and adequate blood clotting during treatment with the FVIII protein without an Fc portion, and wherein administration of the chimeric protein increases BMD and prophylactically treats bleeding episodes in the subject.

Certain aspects of the present disclosure are directed to a method of increasing bone mineral density (BMD) and prophylactically treating bleeding episodes in a subject who has hemophilia A and is being treated with a non-factor replacement protein, the method comprising discontinuing treatment with the non-factor replacement protein and administering to the subject a therapeutically effective amount of a chimeric protein comprising a recombinant FVIII protein and a Fc domain (rFVII1Fc), wherein the subject has been identified as having low BMD and adequate blood clotting during treatment with the non-factor replacement protein, and wherein administration of the chimeric protein increases BMD and prophylactically treats bleeding episodes in the subject.

In some embodiments, the subject has been previously treated to reduce bleeding associated with hemophilia A using a Factor VIII protein without an Fc portion.

In some embodiments, the Factor VIII protein without an Fc portion is PEGylated FVIII that is not fused to a Fc domain. Examples of PEGylated Factor VIII molecules without an Fc portion include, but are not limited to, ADYNOVATE®, ESPEROCT®, and JIVI®.

In some embodiments, the Factor VIII protein without an Fc portion is single-chain FVIII that is not fused to a Fc domain. Examples of single-chain Factor VIII molecules without an Fc portion include, but are not limited to, AFSTYLA®.

In some embodiments, the Factor VIII protein without an Fc portion is recombinant FVIII that does not comprise a moiety that extends the half-life thereof in humans. Examples of Factor VIII molecules that do not comprise a moiety that extends half-life in humans include, but are not limited to, ADVATE®, XYNTHA®, NOVOEIGHt®, and KOVALTRY®.

In some embodiments, the Factor VIII protein without an Fc portion is blood-derived FVIII or plasma-derived FVIII.

In some embodiments, the Factor VIII protein without an Fc portion is damoctocog alfa pegol, turoctocog alfa pegol, turoctocog alfa, lonoctocog alfa, simoctocog alfa, rurioctocog alfa pegol, or octocog alfa.

In some embodiments, the subject has been previously treated to reduce bleeding associated with hemophilia A using a non-factor replacement protein.

In some embodiments, the non-factor replacement protein is emicizumab.

In some embodiments, the emicizumab is emicizumab-kxwh.

In some embodiments, the subject had adequate blood clotting during treatment with the Factor VIII protein without an Fc portion or the non-factor replacement protein.

In some embodiments, the subject has low BMD at a bone site and/or joint where bleeding has not been detected.

### 6. Formulations

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the composition to a child. In some embodiments, the composition is administered to a subject by a healthcare practitioner such as a medical doctor, a medic, or a nurse.

The term “parenteral” as used herein includes subcutaneous, intradermal, intravascular (e.g., intravenous), intramuscular, spinal, intracranial, intrathecal, intraocular, periocular, intraorbital, intrasynovial and intraperitoneal injection or infusion, as well as any similar injection or infusion technique. The composition can be also for example a suspension, emulsion, sustained release formulation, cream, gel or powder. The composition can be formulated as a suppository, with traditional binders and carriers such as triglycerides.

In an example, the pharmaceutical formulation is a liquid formulation, e.g., a buffered, isotonic, aqueous solution. In an example, the pharmaceutical composition has a pH that is physiologic, or close to physiologic. In an example, the aqueous formulation has a physiologic or close to physiologic osmolarity and salinity. In an example, the aqueous formulation can contain sodium chloride and/or sodium acetate.

In some embodiments, the chimeric protein comprising a FVIII and an Fc region used in the methods of the present invention is formulated in a pharmaceutical composition comprising: (a) the chimeric polypeptide; (b) one or more stabilizing agents selected from sucrose, trehalose, raffinose, arginine, or mixture thereof; (c) sodium chloride (NaCl); (d) L-histidine; (e) calcium chloride; and (f) polysorbate 20 or polysorbate 80. In certain embodiments, the pharmaceutical composition comprises: (a) 50 IU/ml to 2500 IU/ml of the chimeric polypeptide; (b) 10 mg/ml to 25 mg/ml of sucrose; (c) 8.8 mg/ml to 14.6 mg/ml sodium chloride (NaCl); (d) 0.75 mg/ml to 2.25 mg/ml L-histidine; (e) 0.75 mg/ml to 1.5 mg/ml calcium chloride dihydrate; and (f) 0.08 mg/ml to 0.25 mg/ml polysorbate 20 or polysorbate 80. In some examples, the pharmaceutical composition used in the methods of the present disclosure is lyophilized.

This disclosure also provides the components of a pharmaceutical kit. Such a kit includes one or more containers and optional attachments. A kit as provided herein facilitates administration of an effective amount of the chimeric protein (e.g., rFVII1Fc) to a subject in need thereof. In certain embodiments, the kit facilitates administration of the chimeric protein (e.g., rFVII1Fc) via intravenous infusion. In certain embodiments, the kit facilitates self-administration of the chimeric protein (e.g., rFVII1Fc) via intravenous infusion.

In some embodiments, the disclosure provides a pharmaceutical kit comprising: a first container comprising a lyophilized powder or cake, where the powder or cake comprises: (i) the chimeric protein (e.g., rFVII1Fc), (ii) sucrose (and/or trehalose, raffinose or arginine); (iii) NaCl; (iv) L-histidine; (v) calcium chloride dihydrate; and (vi) polysorbate 20 or polysorbate 80; and a second container comprising a diluent, e.g., sterilized water for injection, to be combined with the lyophilized powder of the first container. In some embodiments, sufficient diluent is provided to produce about 3 ml of the chimeric protein (e.g., rFVIIIFc) formulation with desired properties as disclosed herein. In some embodiments, the second container is a pre-filled syringe associated with a plunger, to allow addition of the diluent to the first container, reconstitution of the contents of the first container, and transfer back into the syringe. In some embodiments, the kit further provides an adaptor for attaching the syringe to the first container. In some embodiments the kit further provides a needle and infusion tubing, to be attached to the syringe containing the reconstituted FVIII polypeptide (e.g., rFVIIIFc) formulation to allow IV infusion of the formulation.

In some embodiments the chimeric protein (e.g., rFVIIIFc) is provided in a total amount from about 200 IU to about 6000 IU, e.g., about 250 IU, about 500 IU, about 750 IU, about 1000 IU, about 1500 IU, about 2000 IU, about 3000 IU, about 4000 IU, about 5000 IU, or about 6000 IU.

The FVIII portion in the clotting factor or the chimeric protein used herein has FVIII activity. FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is used in conjunction with prothrombin time (PT), which measures the extrinsic pathway.

ROTEM analysis provides information on the whole kinetics of hemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary hemostasis.

The chromogenic assay mechanism is based on the principles of the blood coagulation cascade, where activated FVIII accelerates the conversion of Factor X into Factor Xa in the presence of activated Factor IX, phospholipids and calcium ions. The Factor Xa activity is assessed by hydrolysis of a p-nitroanilide (pNA) substrate specific to Factor Xa. The initial rate of release of p-nitroaniline measured at 405 nmis directly proportional to the Factor Xa activity and thus to the FVIII activity in the sample.

The chromogenic assay is recommended by the FVIII and Factor IX Subcommittee of the Scientific and Standardization Committee (SSC) of the International Society on Thrombosis and Hemostasis (ISTH). Since 1994, the chromogenic assay has also been the reference method of the European Pharmacopoeia for the assignment of FVIII concentrate potency. Thus, in one embodiment, the chimeric protein comprising FVIII has FVIII activity comparable to a chimeric protein comprising mature FVIII or a BDD FVIII (e.g., ADVATE®, REFACTO®, or ELOCTATE®).

In certain embodiments, the effective amount or the effective dose is administered as a single dose. In some embodiments, the effective amount or the effective dose is administered in two or more doses throughout a day.

Having now described the present disclosure in detail, the same will be more clearly understood by reference to the following examples, which are included herewith for purposes of illustration only and are not intended to be limiting of the disclosure. All patents, publications, and articles referred to herein are expressly and specifically incorporated herein by reference.

### EXAMPLES

The present disclosure provides, inter alia, compositions, compounds, kits, and methods for treating subjects with hemophilia A and low BMD, and is not limited by any particular scientific theory.

### Example 1: Recombinant Factor VIII Fc Fusion Protein (rFVII1Fc) Negatively Regulates Inflammatory Osteoclast Formation In Vitro

Decrease in bone mineral density observed in severe hemophilia A (HemA) patients suggests that the absence of FVIII activity and related bleeding episodes have profound effect on bone homeostasis.

Without being bound by any scientific theory, it was hypothesized that the pro-inflammatory milieu in these patients may contribute to exacerbated monocyte/macrophage-derived osteoclastogenesis and subsequent bone erosion, similarly to events reported in case of arthritis-related osteoporosis. The effect of rFVIII vs. rFVII1Fc treatment on monocyte-derived osteoclastogenesis was investigated to determine whether rFVIIIFc inhibits pro-inflammatory osteoclast formation by upregulating the antioxidant NRF2 pathway.

To test this hypothesis, human monocytes from peripheral blood mononuclear cells (PBMC) were isolated and cultured with rhM-CSF and rhRANKL to achieve osteoclast formation, untreated or in the presence of hIgG1, rFVIII or rFVIIIFc. Gene expression changes triggered by the treatments were measured by Q-PCR. Osteoclast phenotype was followed by tartrate-resistant acid phosphatase (TRAP) staining and observing multinucleation. Function of the treated osteoclasts was examined using bone resorption assay.

Total RNA was isolated from macrophages using RNeasy Mini Kit (Qiagen, Valencia, Calif.) and reverse transcribed using SuperScript III Vilo Kit (Thermo Fisher Scientific). Quantitative real-time polymerase chain reaction (PCR) assays were performed using Taqman gene expression assays from Thermo Fisher Scientific and run on a 7500 Fast instrument. The comparative cycle threshold method was used to quantify transcripts relative to the endogenous control gene 36B4.

Human monocyte-derived macrophages were generated from CD14⁺ monocytes isolated from peripheral blood mononuclear cells of healthy human donors.

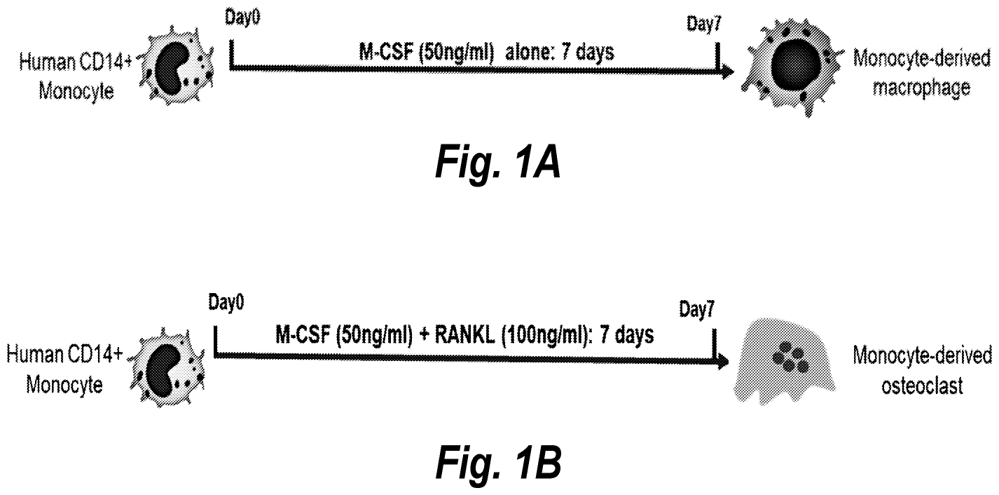

Purified CD14⁺ monocytes were plated in RPMI 1640 Glutamax medium (Thermo Fisher Scientific) supplemented with penicillin, streptomycin, and 10% fetal bovine serum. Monocytes were treated for the duration of the 7 day culture period with human IgG1, B-domain deleted rFVIII, rFVII1Fc (25 nM each) or vehicle (PBS) unless described otherwise. Treatment concentrations were determined in preliminary experiments. A mutant form of rFVIIIFc molecule, rFVII1Fc N297A, which is unable to bind to the FcγRs, was also used in some experiments to determine the effect of the Fc portion. Krishnamoorthy S, et al. Cell Immunol. 2016; 301:30-39. A schematic of the design of the study is shown in FIG. 1.

Results

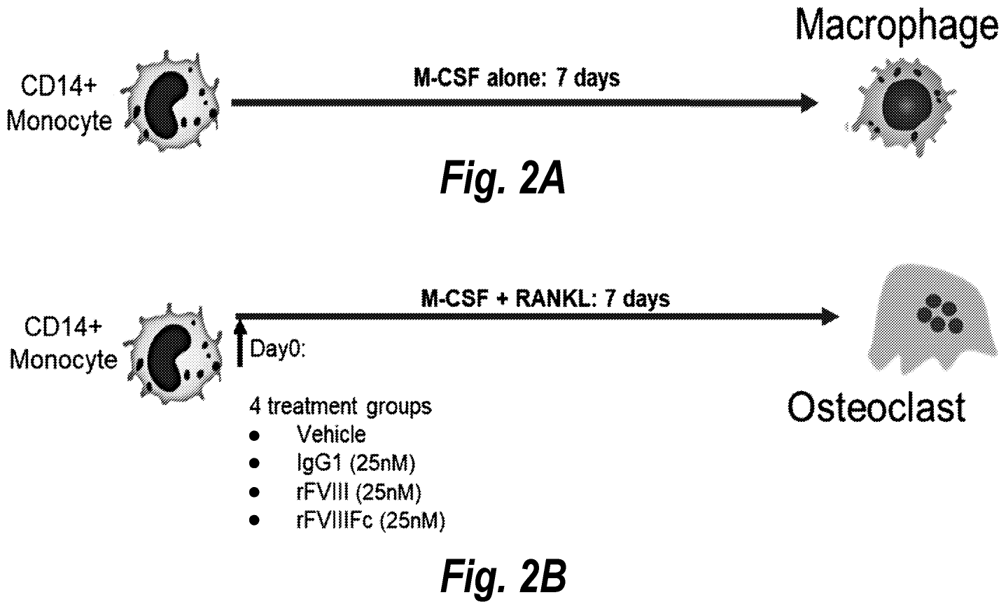

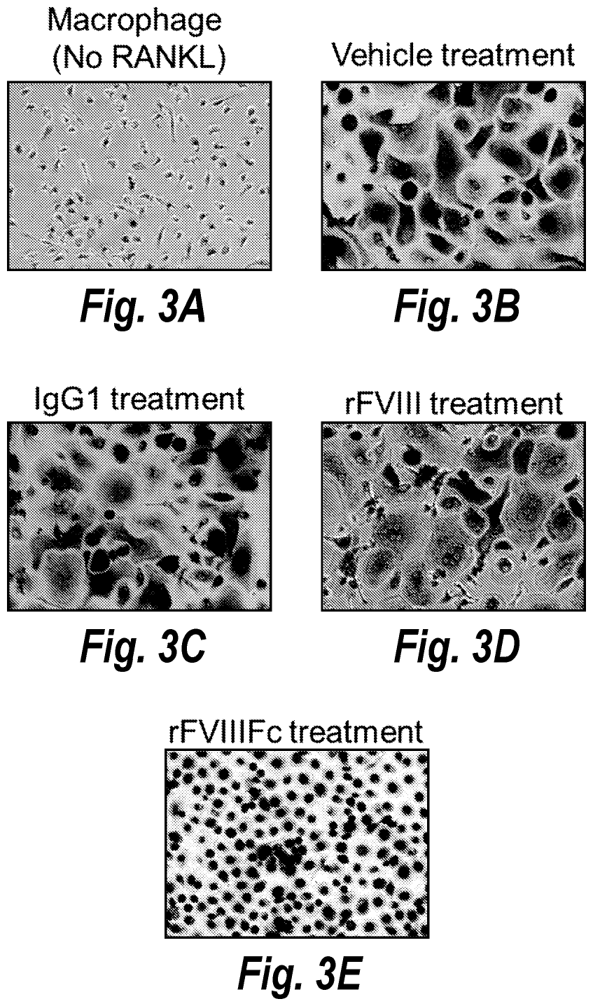

CD14⁺ monocytes were either cultured for 7 days in the presence of M-CSF alone, or treated with one of 4 treatment groups at Day 0 and cultured in the presence of M-CSF and RANKL for 7 days (FIG. 2). Cells of each treatment group were then observed for morphological characteristics by TRAP staining (FIG. 3). Control cells treated without RANKL exhibited distinct macrophage morphology (FIG. 3A). Cells treated with vehicle (FIG. 3B), IgG1 alone (FIG. 3C), or rFVIII alone (FIG. 3D) and cultured with M-CSF and RANKL exhibited large, multinucleated cell bodies characteristic of osteoclasts. Cells treated with rFVII1Fc (FIG. 3E) remained small and contained a single nucleus, indicating that rFVII1Fc treatment inhibited the formation of multinucleated osteoclasts.

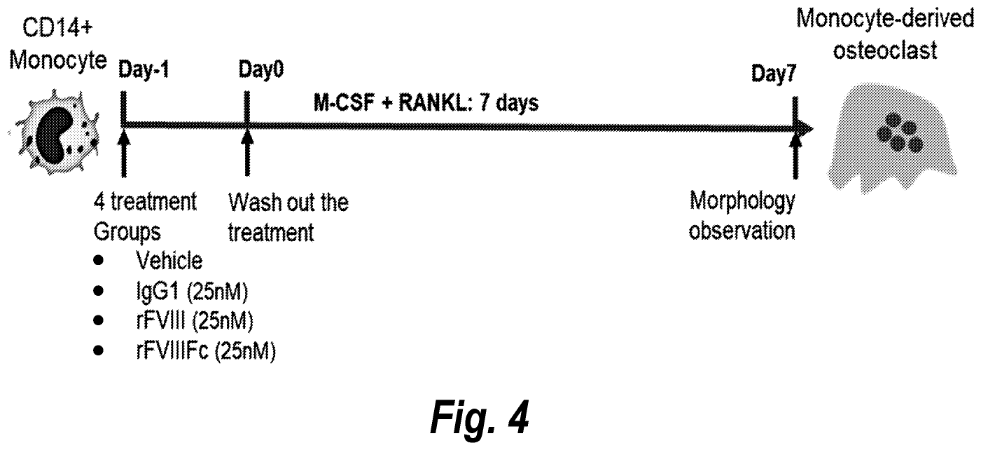

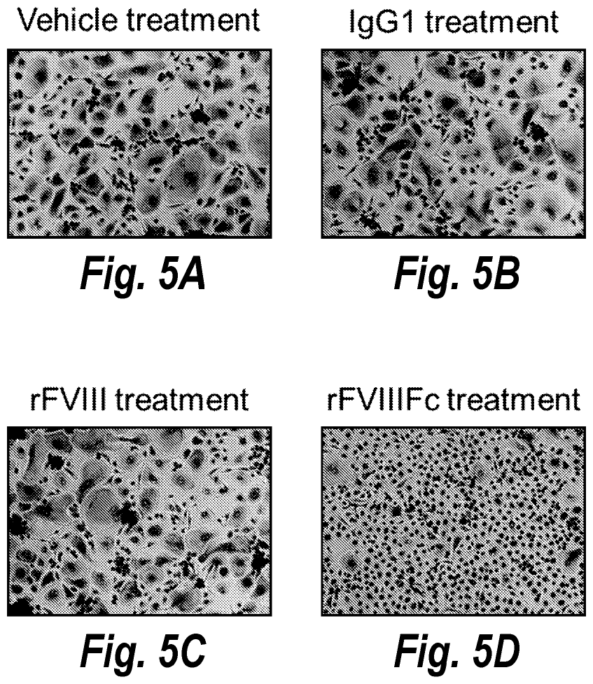

To examine the effect of treatment timing on osteoclastogenesis, CD14⁺ monocytes were treated at day −1 with one of four treatments. After treatment for 24 hours, culture media was removed, cells were centrifuged and washed once with DPBS and resuspended in culture media containing M-CSF and RANKL and replated (FIG. 4). CD14⁺ monocytes treated with vehicle (FIG. 5A), IgG1 alone (FIG. 5B), or rFVIII alone (FIG. 5C) on day −1, washed out at day 0, cultured with M-CSF and RANKL and examined by TRAP staining differentiated into large, multinucleated cells characteristic of osteoclast morphology. CD14⁺ monocytes treated similarly with rFVIIIFc (FIG. 5D) and examined by TRAP staining, did not differentiate into osteoclasts, as very few of the characteristic large multinucleated cells were observed, indicating that rFVII1Fc treatment of monocytes for only one day substantially inhibited the formation of osteoclast cells in vitro after 7 days differentiation. This is physiologically relevant to both FVIII and monocytes' blood circulatory properties, as rFVII1Fc is only expected to interact with monocytes in blood circulation. rFVII1Fc treatment showed no detectable effects on completely differentiated monocyte-derived osteoclasts (data not shown).

Summary

Monocyte-derived osteoclast development was significantly impaired in the presence of rFVII1Fc. According to morphology observations, treatment of monocytes with rFVII1Fc for only one day was sufficient to inhibit formation of osteoclast cells.

### Example 2: rFVII1Fc Inhibits Bone Resorption Activity of Osteoclasts In Vitro

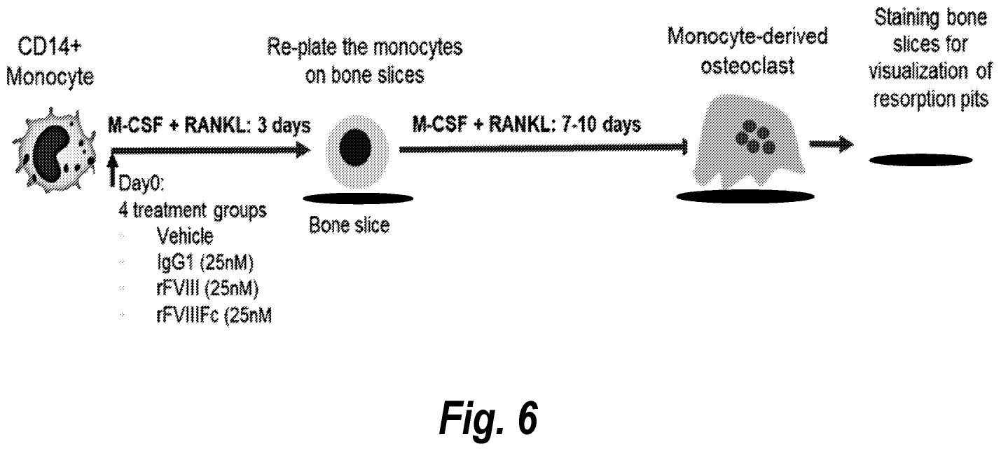

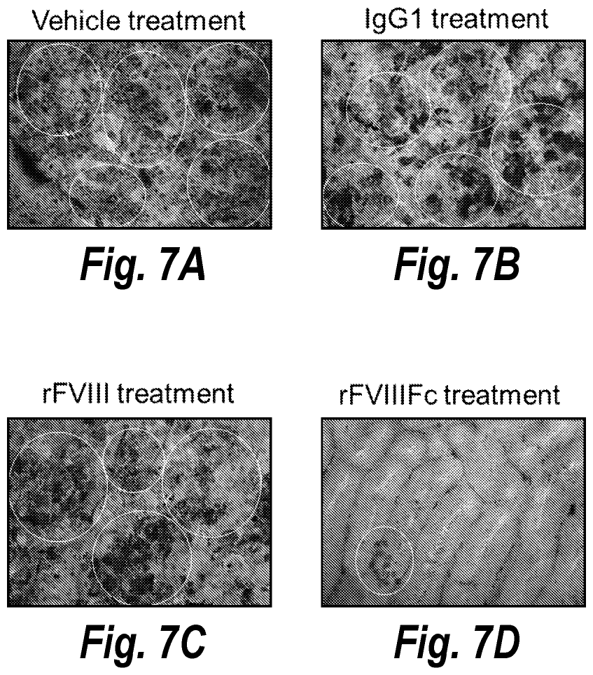

As rFVII1Fc was able to inhibit osteoclast formation, we next examined the effect of rFVII1Fc on the bone resorption activity of osteoclasts. CD14⁺ monocytes were treated with vehicle, IgG1 alone, rFVIII alone, or rFVII1Fc on day 0 and cultured in the presence of M-CSF and RANKL for 3 days. On day 3, monocytes were re-plated on bovine cortical bone slices and co-cultured in the presence of M-CSF and RANKL for 7-10 days. After the 7-10 day coculture period, monocyte-derived cells were removed and bone slices were examined by toluidine blue staining (FIG. 6). Bone slices co-cultured with vehicle (FIG. 7A), IgG1 (FIG. 7B), or rFVIII (FIG. 7C) treated monocytes displayed clear bone resorption (FIGS. 7A, 7B, 7C; circled regions), indicating osteoclasts derived from this treatment pool were still able to actively break down bone. Bone slices co-cultured with rFVII1Fc treated monocytes (FIG. 7D) displayed noticeably less bone resorption (FIG. 7D, circled areas) when compared to the three control groups, suggesting that rFVII1Fc treatment of monocytes at day 0 substantially inhibits the bone resorption activity of the cells after 7-10 days of differentiation.

Summary

rFVII1Fc treatment of monocytes cultured with osteoclast differentiation factors (M-CSF and RANKL) leads to decreased bone resorption activity of the treated cells.

### Example 3: Effects of rFVIIIFc on Gene Expression and Function in Osteoclastogenesis

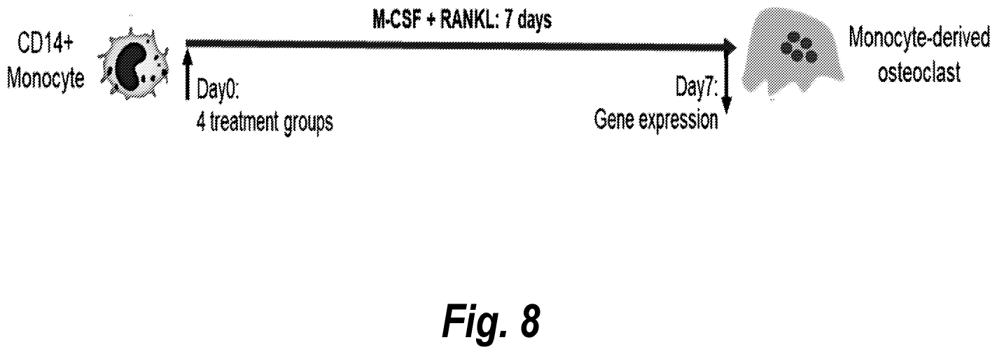

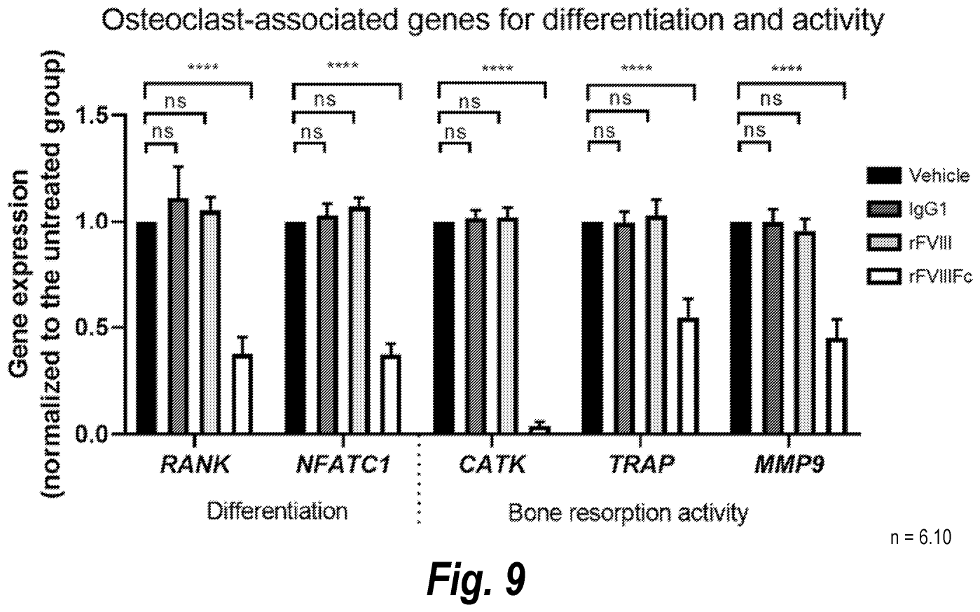

We next investigated whether the reduced osteoclast activity and morphology of rFVII1Fc corresponded with a decrease in osteoclast related genes. CD14⁺ monocytes were treated with vehicle, IgG1 alone, rFVIII alone or rFVII1Fc at day 0 and cultured in the presence of M-CSF and RANKL for 7 days. Cells were then harvested, RNA extracted, and gene expression levels quantified by quantitative real-time PCR (FIG. 8). Osteoclast-associated genes were then measured in vehicle treated (FIG. 9, black bars), IgG1 treated (FIG. 9, dark gray bars), rFVIII treated (FIG. 9, light gray bars) and rFVII1Fc treated (FIG. 9, white bars) cells, and normalized to the expression level of the vehicle treated group. Markers of osteoclast differentiation (FIG. 9, RANK, NFATC1) and bone resorption activity (FIG. 9, CATK, TRAP, MMP9) were analyzed. No significant change was observed between the vehicle, IgG1, or rFVIII treated groups for any of the genes analyzed. However, significant decreases in gene expression were observed for rFVII1Fc treated cells in both markers of osteoclast differentiation (RANK, NFATC1) and activity (CATK, TRAP, MMP9) when compared to the other treatment groups. See FIG. 9, white bars.

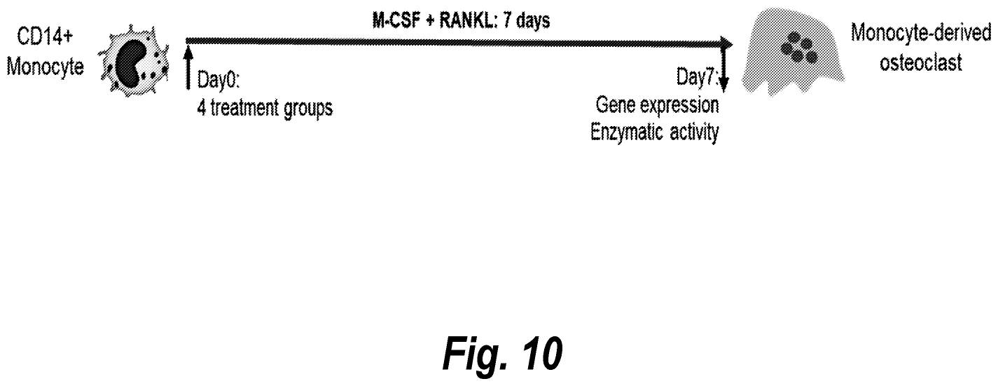

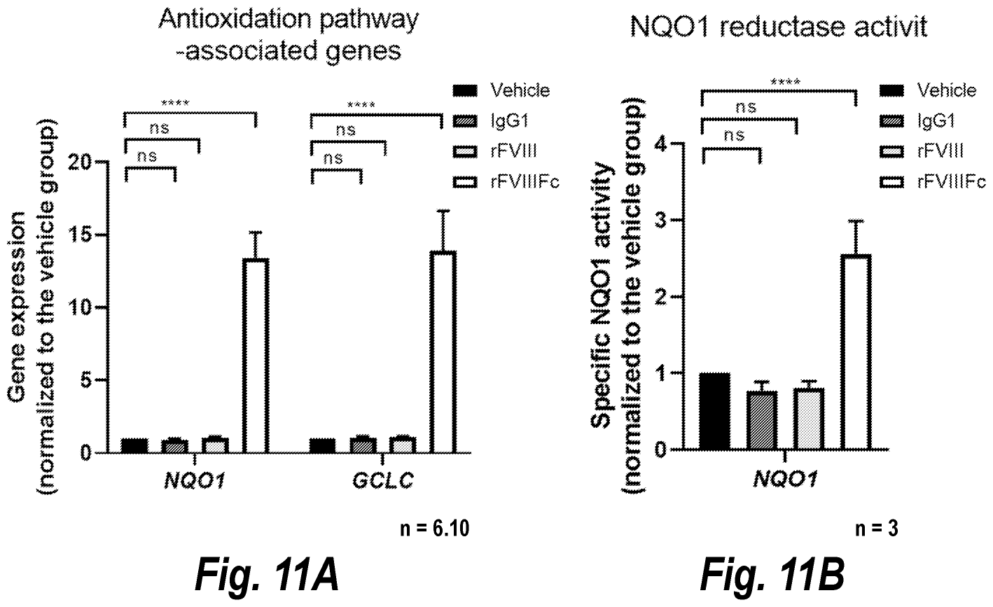

We next investigated the response of NRF2-related genes during osteoclastogenesis in the 4 treatment groups described above. NRF2 is known to play a role in regulating antioxidation pathways that are downregulated during osteoclastogenesis (Kanzaki J Biol Chem). NRF2 controls expression of cryoprotective enzymes such as GCLC and NQO1. CD14⁺ monocytes were treated with vehicle, IgG1 alone, rFVIII alone or rFVII1Fc at day 0 and cultured in the presence of M-CSF and RANKL for 7 days. Cells were then harvested, RNA extracted, and gene expression levels quantified by quantitative real-time PCR (FIG. 10). Expression of NRF2-controlled genes NQO1 and GCLC was not significantly altered in monocytes treated with IgG1 alone (FIG. 11A, dark gray bars) or rFVIII alone (FIG. 11A, light gray bars) when compared to vehicle treated cells (FIG. 11A, black bars). However, monocytes treated with rFVII1Fc exhibited a significant increase in expression of both NQO1 and GCLC compared to the vehicle treated group. See FIG. 11A, white bars.

We next investigated NQO1 reductase activity in vehicle (FIG. 11B, black bars), IgG1 alone (FIG. 11B, dark gray bars), rFVIII alone (FIG. 11B, light gray bars) or rFVII1Fc treated (FIG. 11B, white bars) monocytes. Compared to the vehicle treated group, neither IgG1 alone or rFVIII exhibited a significant increase in specific NQO1 activity (FIG. 11B). However, specific NQO1 activity was significantly increased in rFVII1Fc treated cells, indicating that regulation of this important pathway may play a role in rFVII1Fc mediated inhibition of osteoclastogenesis.

Summary

Gene and protein expression of rFVII1Fc-treated cells showed upregulation of the antioxidant NRF2 pathway and downregulation of osteoclast-specific markers and genes known to have a role in osteoclast formation and bone resorption. Conversely, increases in cryoprotective enzymes (NQO1, GCLC) were observed in the rFVII1Fc-treated osteoclasts as compared to the untreated, IgG1 alone, or rFVIII-treated cells.

### Example 4: Role of Fc Portion of rFVIIIFc in Inhibition of Osteoclastogenesis

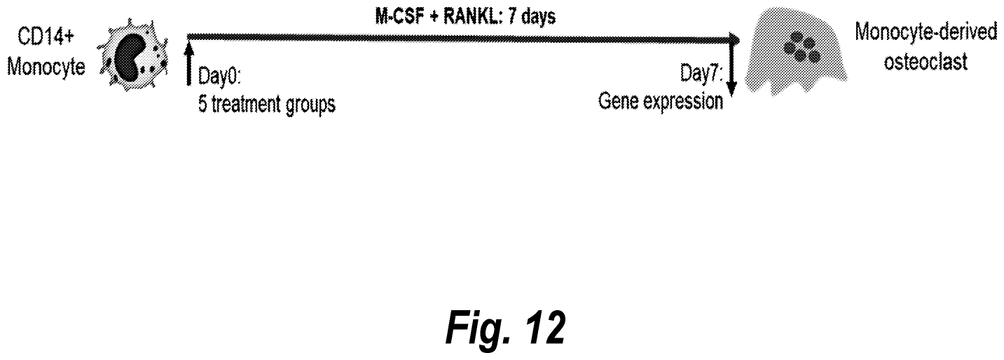

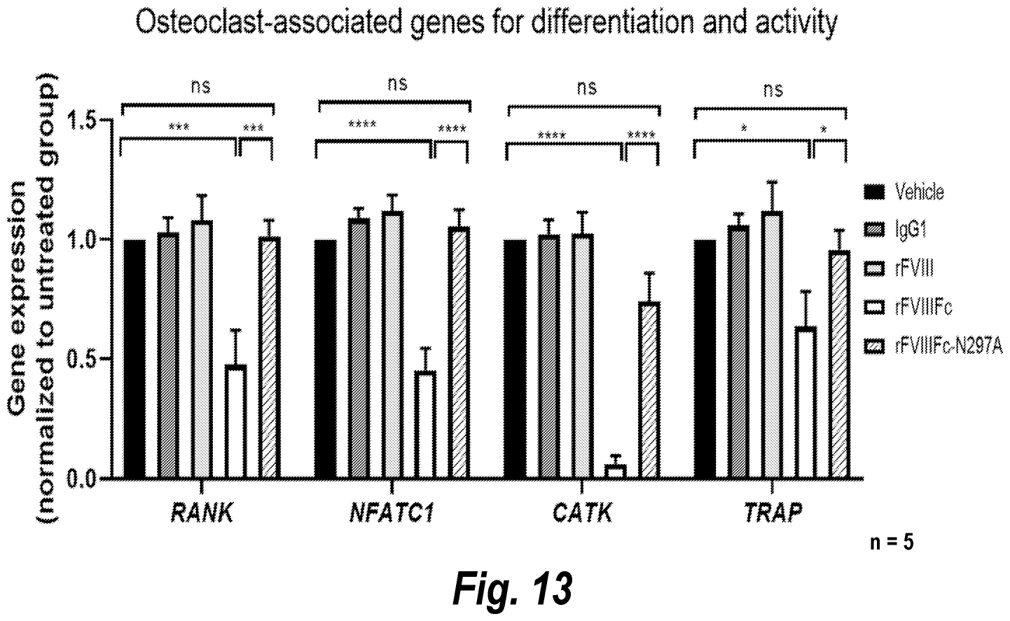

We next investigated the role of the Fc portion of rFVII1Fc in inhibition of osteoclastogenesis. CD14⁺ monocytes were treated with vehicle, IgG1 alone, rFVIII alone, rFVIIIFc, and rFVIIIFc-N297A (unable to bind to FcγRs) at day 0 and cultured in the presence of M-CSF and RANKL for 7 days. Cells were then harvested, RNA extracted, and gene expression levels quantified by quantitative real-time PCR (FIG. 12). Markers of osteoclast differentiation (RANK, NFATC1) and activity (CATK, TRAP) were measured and normalized to the vehicle treated group (FIG. 13, black bars). Neither IgG1 alone (FIG. 13, dark gray bars) or rFVIII alone (FIG. 13, light gray bars) treated cells displayed any significant change in gene expression compared to the vehicle treated group. rFVII1Fc treated cells (FIG. 13, white bars) exhibited a significant decrease in all osteoclast related markers. This reduction was not observed when FcγRs binding was abolished in the rFVII1Fc-N297A treated group (FIG. 13, diagonal lined bars).

Summary

The inhibitory effects of rFVII1Fc on monocyte-derived osteoclast formation and osteoclast-specific gene expression require the Fc domain and FcγRs interaction.

### Example 5: Dose Dependent Differentiation of Monocytes Treated with rFVIIIFc

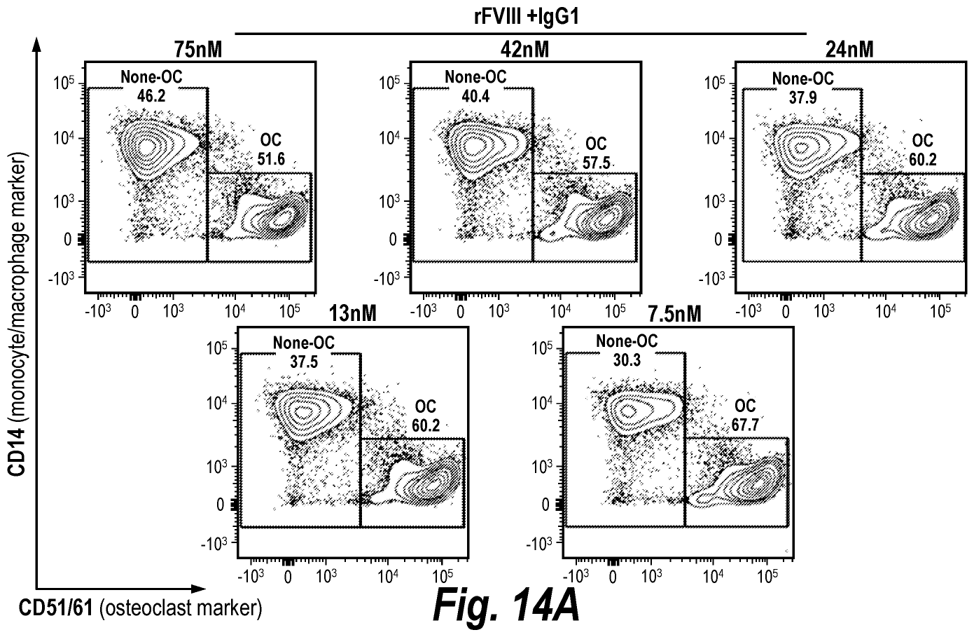

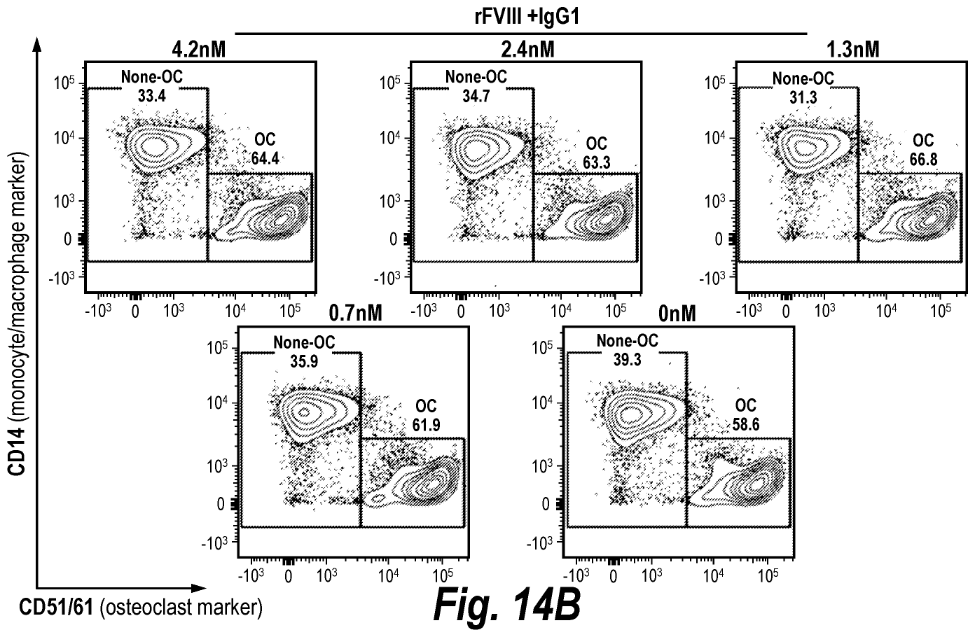

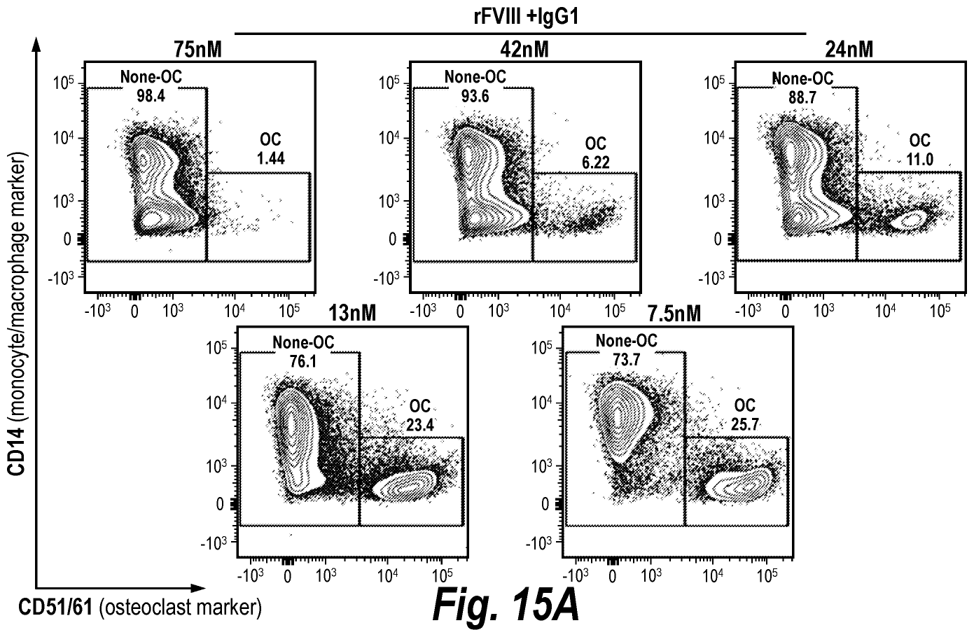

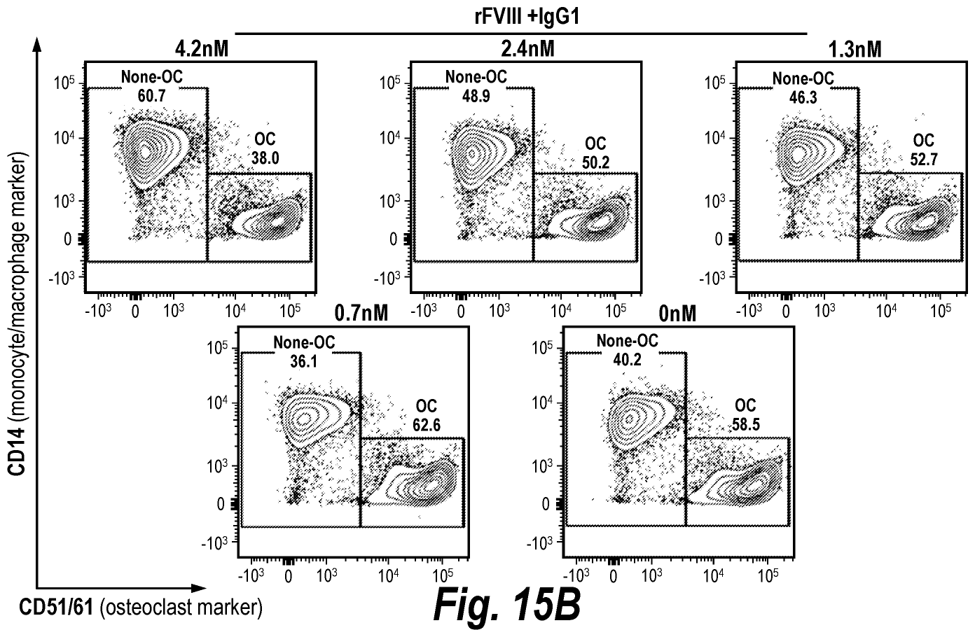

We investigated the effect of dosage of rFVII1Fc on immunophenotype of MCSF/RANKL-differentiated monocytes. CD14⁺ monocytes were treated with a dose (75 nM, 42 nM, 24 nM, 13 nM, 7.5 nM, 4.2 nM, 2.4 nM, 1.3 nM, 0.7 nM or 0 nM) rFVIII+IgG1 (FIGS. 14A-14B) or rFVII1Fc (FIGS. 15A-15B) at day 0 and cultured in the presence of M-CSF and RANKL for 7 days. Cells were then harvested, stained with fluorescent monoclonal antibodies, and subjected to acquisition by flow cytometer. Cells were stained with fluorescent antibodies against CD14 (monocyte/macrophage marker) and CD51/61 (osteoclast marker), as well as other monocyte/macrophage markers CD16, CD32, CD64, CD163, CD33, CD35, CD44, CD11b, and CD172ab. Osteoclasts are characterized as CD51/61 high cells in conjunction with low expression of CD14.

Summary

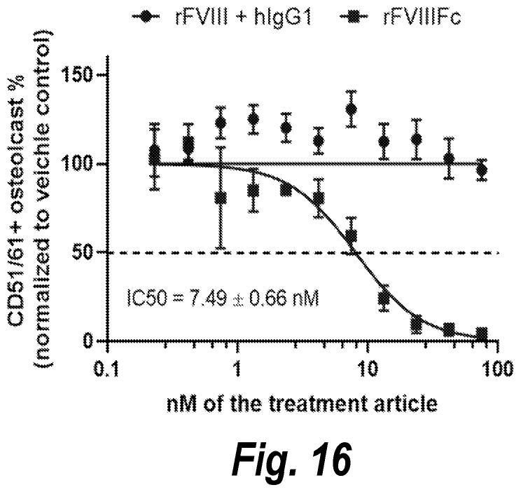

Treatment with a rFVIII+IgG1 exhibited only a minor effect on the inhibition of osteoclast formation, as 51.6% of cells treated with a 75 nM dose of rFVIII+IgG1 differentiated into osteoclasts (CD51/61^{high}/CD14^{low}; FIG. 14A), compared to 61.9% of cells treated with 0.7 nM of rFVIII+IgG1, or 58.6% of cells treated with vehicle (FIG. 14B). Conversely, treatment with rFVII1Fc exhibited a substantial inhibition of osteoclast formation. Only 1.44% of cells treated with 75 nM rFVII1Fc differentiated into osteoclasts (FIG. 15A), compared to 62.6% of cells treated with only 0.7 nM rFVII1Fc or 58.5% of cells treated with vehicle (FIG. 15B). Additionally, higher doses of rFVII1Fc treatment revealed a distinct (CD51/61^{negative}CD14^{low}) immunophenotype among treated cells (FIG. 15A). A mean IC50 of 7.49 nM (±0.66 nM, n=3) was observed with respect to the inhibitory effect of osteoclast formation on cells treated with rFVII1Fc (FIG. 16) when normalized to vehicle control.

### Example 6: Fcγ Receptor Mediated Inhibition of Osteoclastogenesis

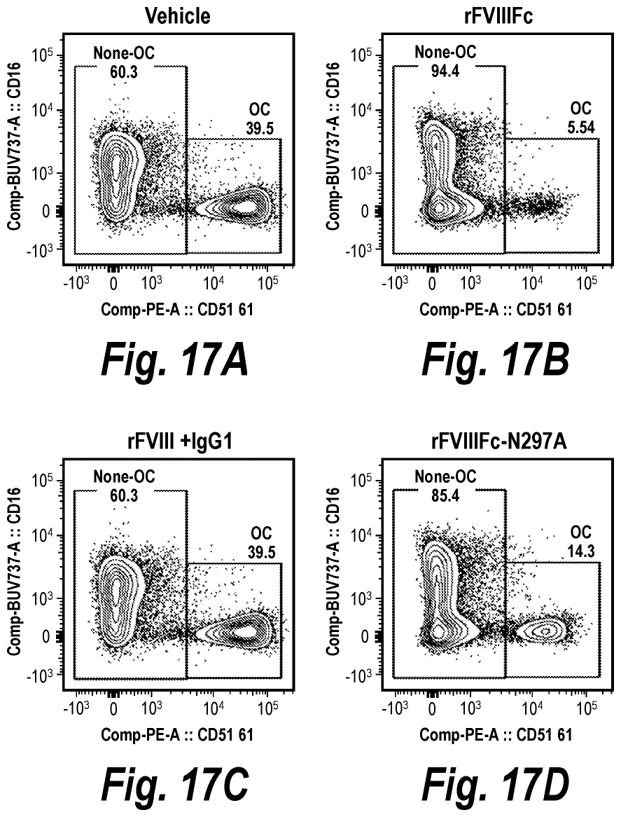

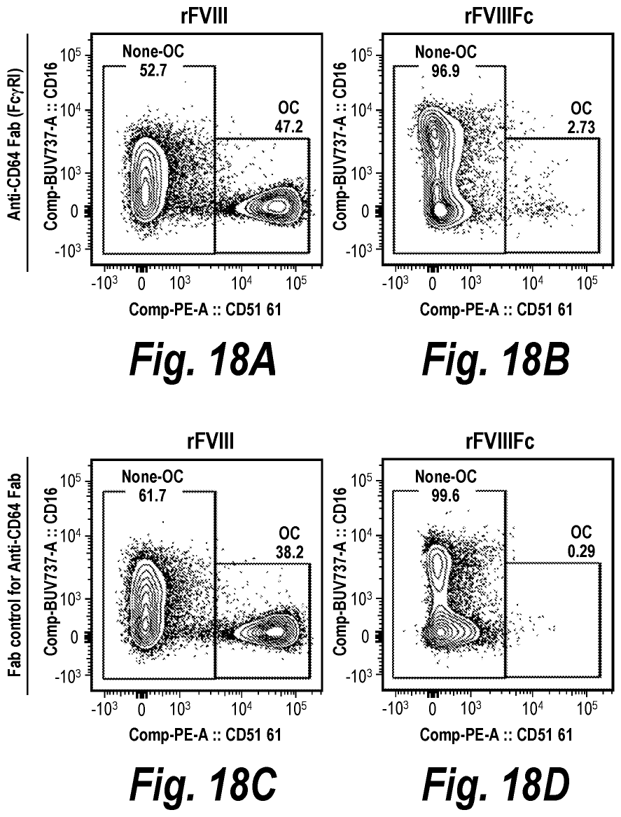

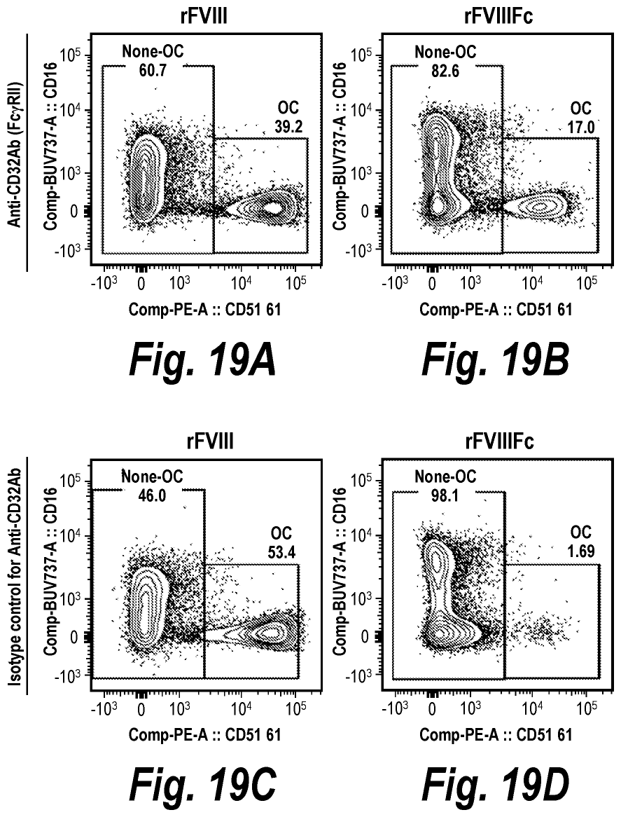

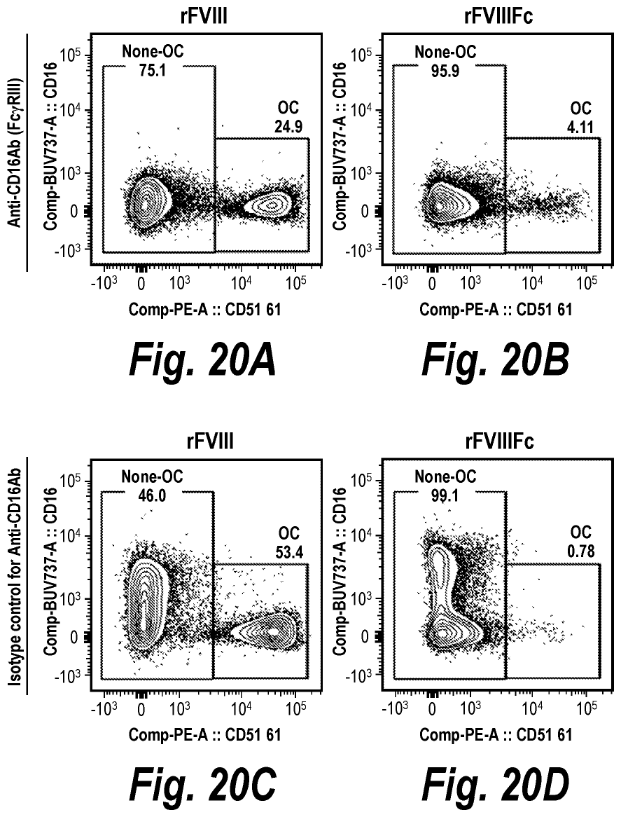

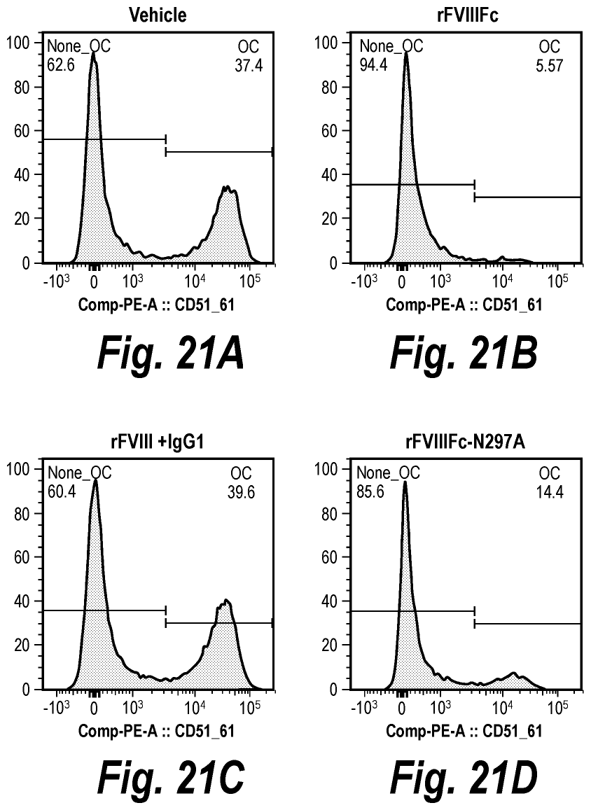

We next investigated the role of the Fcγ receptors in the inhibition of osteoclastogenesis from monocytes treated with rFVIIIFc. In a first experiment, CD14⁺ monocytes were treated with vehicle (FIG. 17A, FIG. 21A), rFVII1Fc (FIG. 17B, FIG. 21B), rFVIII+IgG1 (FIG. 17C, FIG. 21C), or rFVII1Fc-N297A (FIG. 17D, FIG. 21D; unable to bind to FcγRs) at day 0 and cultured in the presence of M-CSF and RANKL for 7 days. Cells were then harvested, stained with fluorescent monoclonal antibodies, and subjected to acquisition by flow cytometry. Cells were stained with fluorescent antibodies against CD16 (monocyte/macrophage marker) and CD51/61 (osteoclast marker). In a second experiment, at day 0, CD14⁺ monocytes were first treated by a blocking antibody against FcγR1 (Anti-CD64 antibody Fab, FIG. 18), FcγR2 (Anti-CD32 antibody, FIG. 19), or FcγR3 (Anti-CD16 antibody, FIG. 20) or each corresponding isotype control antibody, then treated with rFVII1Fc or rFVIII, and then further cultured in the presence of M-CSF and RANKL for 7 days. Cells were then harvested, stained with fluorescent monoclonal antibodies, and subjected to acquisition by flow cytometer. Cells were stained with fluorescent antibodies against CD16 (monocyte/macrophage marker) and CD51/61 (osteoclast marker).

Summary

39.5% of both vehicle treated cells (FIG. 17A; 37.4% FIG. 21A) and cells treated with rFVIII+IgG1 (FIG. 17C; 39.6% FIG. 21C) were characterized as CD51/61^{high} osteoclasts, while only 5.54% of cells treated with rFVII1Fc (FIG. 17B; 5.57% FIG. 21B) were characterized as CD51/61^{high} osteoclasts. Ablation of the interaction between the Fc domain and Fcγ receptors by mutation of N297 (rFVII1Fc-N297A) resulted in a partial rescue of osteoclast formation (FIG. 17D, FIG. 21D).

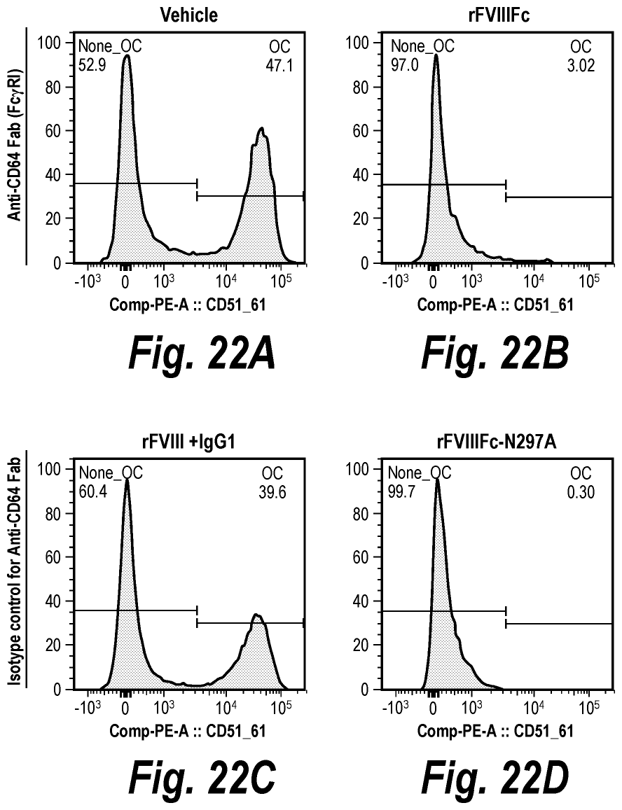

47.2% of cells treated with rFVIII and an antibody Fab to block FcγR1 interactions (Anti-CD64 antibody, FIG. 18A; 47.1% FIG. 22A) were characterized as osteoclasts, compared to 2.73% of cells treated with rFVII1Fc and an Anti-CD64 antibody Fab (FIG. 18B; 3.02% FIG. 22B). This pattern persisted in cells treated with a Fab control antibody (rFVIII+control, FIG. 18C, FIG. 22C; rFVII1Fc+control, FIG. 18D, FIG. 22D). This pattern indicates that rFVII1Fc inhibition of osteoclast formation is not likely mediated through interactions with FcγR1 alone.

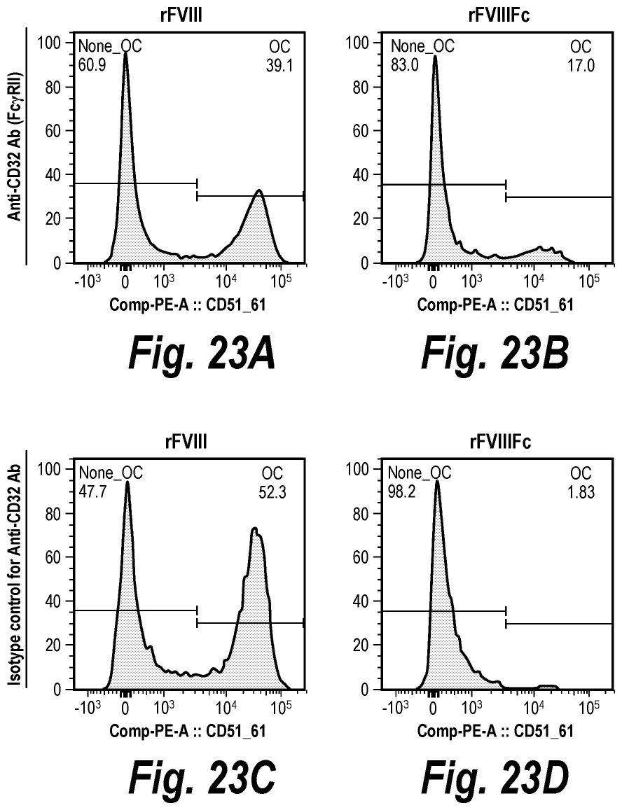

39.2% of cells treated with rFVIII and an antibody to block FcγR2 interactions (Anti-CD32 antibody, FIG. 19A; 39.1% FIG. 23A) were characterized as osteoclasts, compared to 17.0% of cells treated with rFVII1Fc and an Anti-CD32 antibody (FIG. 19B and FIG. 23B). This rescue of osteoclast formation was ablated with treatment of an isotype control (rFVIII+control, FIG. 19C and FIG. 23C; rFVII1Fc+control, FIG. 19D and FIG. 23D). This partial rescue after interactions with FcγR2 are blocked indicates that the inhibition of osteoclast formation is likely controlled by rFVIIIFc interactions with FcγR2.

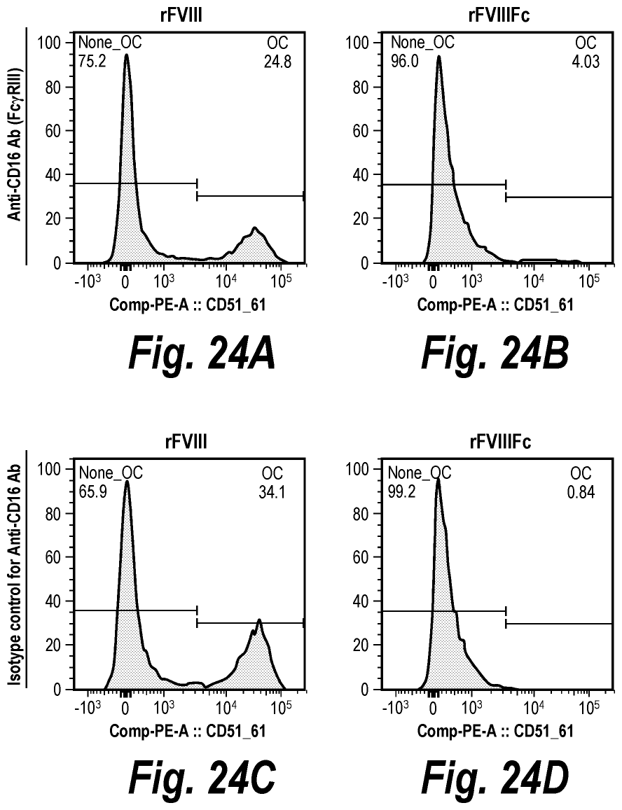

24.9% of cells treated with rFVIII and an antibody to block FcγR3 interactions (Anti-CD16 antibody, FIG. 20A; 24.8% FIG. 24A) were characterized as osteoclasts, compared to 4.11% of cells treated with rFVII1Fc and an Anti-CD16 antibody (FIG. 20B; 4.03% FIG. 24A). This pattern persisted when cells were treated with an isotype control antibody (rFVIII+control, FIG. 20C and FIG. 24C; rFVII1Fc+control, FIG. 20D and FIG. 24D). Without being bound by any scientific theory, this pattern indicates that rFVII1Fc inhibition of osteoclast formation is not likely mediated through interactions with FcγR3 alone.

### Example 7: Role of the FVIII Light Chain in Fcγ Receptor Mediated Inhibition of Osteoclastogenesis

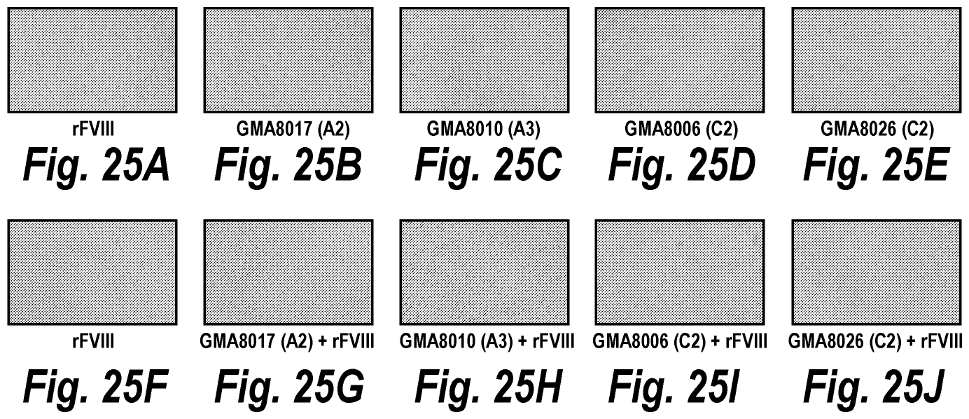

We investigated the role of the C1 and C2 domains of FVIII in the inhibition of osteoclastogenesis from monocytes treated with rFVII1Fc. CD14⁺ monocytes were treated with rFVIII (FIG. 25A), or each alone of monoclonal antibodies targeting the A2 domain of FVIII (GMA8017, FIG. 25B), the A3 domain of FVIII (GMA8010, FIG. 25C), or the C2 domain of FVIII (GMA8006; FIG. 25D; GMA8026, FIG. 25E) at day 0, then cultured in the presence of M-CSF and RANKL for 7 days, and then visualized for osteoclastogenesis. CD14⁺ monocytes were also treated with rFVII1Fc (FIG. 25F), or rFVII1Fc in the presence of each of the monoclonal antibodies targeting the A2 domain of FVIII (GMA8017, FIG. 25G), the A3 domain of FVIII (GMA8010, FIG. 25H), or the C2 domain of FVIII (GMA8006; FIG. 25I; GMA8026, FIG. 25J) at day 0, then cultured in the presence of M-CSF and RANKL for 7 days, and then visualized for osteoclastogenesis. Monocytes treated with rFVIII or antibodies alone (FIGS. 25A-E) displayed characteristic osteoclast morphology after culture for 7 days. When monocytes were treated with rFVII1Fc (FIG. 25F), development of osteoclast morphology was effectively inhibited at day 7, as discussed in previous examples. rFVII1Fc treated monocytes in the presence of the antibodies blocking the A2 domain of FVIII (FIG. 25G) or the A3 domain of FVIII (FIG. 25H) also effectively inhibited the development of osteoclast morphology. However, the rFVII1Fc-dependent inhibition of osteoclastogenesis is reversed in the presence of the antibodies targeting C2 domain of FVIII, (FIGS. 25I, 25J). Monocytes treated with rFVII1Fc and C2 targeting antibodies simultaneously (FIGS. 25I, 25J) exhibited characteristic osteoclast morphology similar to that of the controls after 7 days of culture.

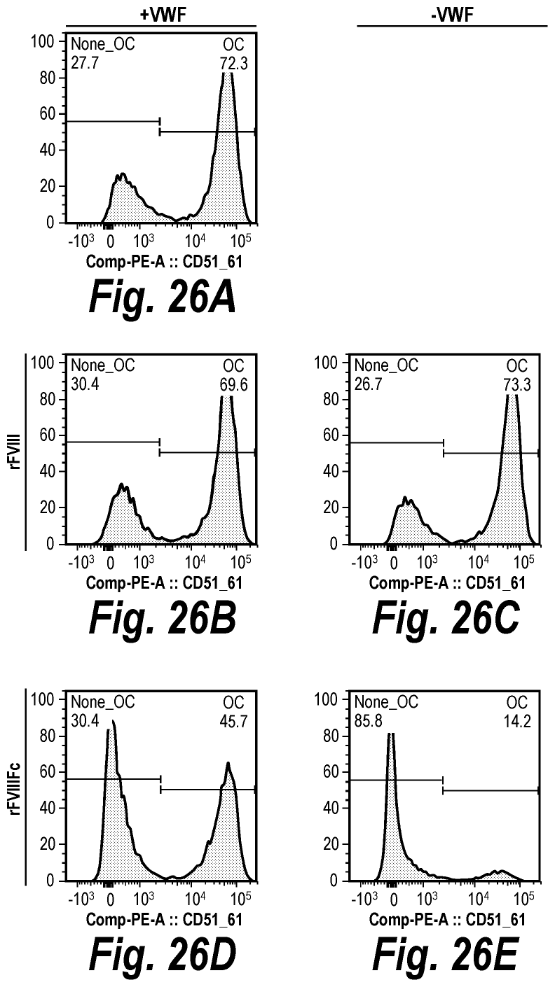

We also investigated osteoclastogenesis in monocytes treated with rFVIII or rFVII1Fc when bound to von Willebrand factor (VWF). When CD14⁺ monocytes were treated with VVVF alone at day 0 (FIG. 26A), 72.3% of cells were characterized as osteoclasts after 7 days in culture in the presence of M-CSF and RANKL. When CD14⁺ monocytes were treated at day 0 with rFVIII in the presence of VVVF (FIG. 26B) or in the absence of VWF (FIG. 26C), the majority of cells (69.6%, and 73.3%) were similarly characterized as osteoclasts after 7 days in culture with M-CSF and RANKL. Together they confirmed that there was no effect of added VWF with rFVIII treatment. When CD14⁺ monocytes were treated at day 0 with rFVII1Fc alone, a much smaller proportion (14.2%) of the cells (FIG. 26E) were characterized as osteoclasts at day 7, consistent with the inhibition of osteoclastogenesis previously discussed. However, this inhibition was partially reversed when cells were treated with both rFVII1Fc and VVVF (FIG. 26D), as an increased proportion (45.7%) of cells were characterized as osteoclasts.

### Example 8: Summary of Role of Fc Portion of rFVIIIFc in Inhibition of Osteoclastogenesis

To study the role of the Fc portion of rFVII1Fc in inhibition of osteoclastogenesis, primary human blood monocytes were treated with rFVII1Fc or rFVIII plus human IgG at various concentrations and then are cultured for osteoclast differentiation in vitro. Multiple myeloid lineage markers were used to immunophenotype and distinguish differentiated monocytes and osteoclasts. The involvement of Fc or FVIII domains in mediating rFVII1Fc interaction with monocytes was probed using antibodies blocking each type of FcγRs, or anti-FVIII antibodies and Von Willebrand factor (VWF) binding to various FVIII domains.

Without being bound by any scientific theory, the results indicated that cells differentiated from the rFVII1Fc-treated monocytes were phenotypically distinct from osteoclasts and remained largely monocytic. For the interaction between rFVII1Fc and monocytes modulating this phenotype, the Fc domain most effectively engaged FcγR2 on the cell surface; C1 and C2 domains of FVIII were mapped to be required for interacting with monocytes, also evidenced by loss of the immune-regulatory effects of VWF-complexed rFVII1Fc.

Without being bound by any scientific theory, these data suggest a “dual-touchpoints” model for rFVII1Fc interacting with monocytes. The FVIII portion interacts with monocytes via C1 and C2 domains and, in parallel, the Fc domain predominantly engages FcγR2 on the same cell, subsequently reducing monocyte differentiation potential into osteoclasts. Therefore, rFVII1Fc may possess a biological activity unique from rFVIII which may reduce joint bone erosion and bone mass loss in patients.

### SEQUENCES

TABLE 1
Exemplary Sequences

| | |
|---|---|
| SEQ ID NO: 1 | ATRRYYLGAVELSWDYMQSDLGELP |
| rFVIIIFc | VDARFPPRVPKSFPFNTSWYKKTLF |
| amino acid | VEFTDHLFNIAKPRPPWMGLLGPTI |
| sequence | QAEVYDTWITLKNMASHPVSLHAVG |
| | VSYWKASEGAEYDDQTSQREKEDDK |
| | VFPGGSHTYVWQVLKENGPMASDPL |
| | CLTYSYLSHVDLVKDLNSGLIGALL |
| | VCREGSLAKEKTQTLHKFILLFAVF |
| | DEGKSWHSETKNSLMQDRDAASARA |
| | WPKMHTVNGYVNRSLPGLIGCHRKS |
| | VYWHVIGMGTTPEVHSIFLEGHTFL |
| | VRNHRQASLEISPITFLTAQTLLMD |
| | LGQFLLFCHISSHQHDGMEAYVKVD |
| | SCPEEPQLRMKNNEEAEDYDDDLTD |
| | SEMDWRFDDDNSPSFIQIRSVAKKH |
| | PKTWVHYIAAEEEDWDYAPLVLAPD |
| | DRSYKSQYLNNGPQRIGRKYKKVRF |
| | MAYTDETFKTREAIQHESGILGPLL |
| | YGEVGDTLLIIFKNQASRPYNIYPH |
| | GITDVRPLYSRRLPKGVKHLKDFPI |
| | LPGEIFKYKWTVTVEDGPTKSDPRC |
| | LTRYYSSFVNMERDLASGLIGPLLI |
| | CYKESVDQRGNQIMSDKRNVILFSV |
| | FDENRSWYLTENIQRFLPNPAGVQL |
| | EDPEFQASNIMHSINGYVFDSLQLS |
| | VCLHEVAYWYILSIGAQTDFLSVFF |
| | SGYTFKHKMVYEDTLTLFPFSGETV |
| | FMSMENPGLWILGCHNSDFRNRGMT |
| | ALLKVSSCDKNTGDYYEDSYEDISA |
| | YLLSKNNAIEPRSFSQNPPVLKRHQ |
| | REITRTTLQSDQEEIDYDDTISVEM |
| | KKEDFDIYDEDENQSPRSFQKKTRH |
| | YFIAAVERLWDYGMSSSPHVLRNRA |
| | QSGSVPQFKKVVFQEFTDGSFTQPL |
| | YRGELNEHLGLLGPYIRAEVEDNIM |
| | VTFRNQASRPYSFYSSLISYEEDQR |
| | QGAEPRKNFVKPNETKTYFWKVQHH |
| | MAPTKDEFDCKAWAYFSDVDLEKDV |
| | HSGLIGPLLVCHTNTLNPAHGRQVT |
| | VQEFALFFTIFDETKSWYFTENMER |
| | NCRAPCNIQMEDPTFKENYRFHAIN |
| | GYIMDTLPGLVMAQDQRIRWYLLSM |
| | GSNENIHSIHFSGHVFTVRKKEEYK |
| | MALYNLYPGVFETVEMLPSKAGIWR |
| | VECLIGEHLHAGMSTLFLVYSNKCQ |
| | TPLGMASGHIRDFQITASGQYGQWA |
| | PKLARLHYSGSINAWSTKEPFSWIK |
| | VDLLAPMIIHGIKTQGARQKFSSLY |
| | ISQFIIMYSLDGKKWQTYRGNSTGT |
| | LMVFFGNVDSSGIKHNIFNPPIIAR |
| | YIRLHPTHYSIRSTLRMELMGCDLN |
| | SCSMPLGMESKAISDAQITASSYFT |
| | NMFATWSPSKARLHLQGRSNAWRPQ |
| | VNNPKEWLQVDFQKTMKVTGVTTQG |
| | VKSLLTSMYVKEFLISSSQDGHQWT |
| | LFFQNGKVKVFQGNQDSFTPVVNSL |
| | DPPLLTRYLRIHPQSWVHQIALRME |
| | VLGCEAQDLYDKTHTCPPCPAPELL |
| | GGPSVFLFPPKPKDTLMISRTPEVT |
| | CVVVDVSHEDPEVKFNWYVDGVEVHN |
| | AKTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPGK |
| SEQ ID NO: 2 | ATRRYYLGAVELSWDYMQSDLGELP |
| FVIII portion of | VDARFPPRVPKSFPFNTSWYKKTLF |
| rFVIIIFc | VEFTDHLFNIAKPRPPWMGLLGPTI |
| | QAEVYDTWITLKNMASHPVSLHAVG |
| | VSYWKASEGAEYDDQTSQREKEDDK |
| | VFPGGSHTYVWQVLKENGPMASDPL |
| | CLTYSYLSHVDLVKDLNSGLIGALL |
| | VCREGSLAKEKTQTLHKFILLFAVF |
| | DEGKSWHSETKNSLMQDRDAASARA |
| | WPKMHTVNGYVNRSLPGLIGCHRKS |
| | VYWHVIGMGTTPEVHSIFLEGHTFL |
| | VRNHRQASLEISPITFLTAQTLLMD |
| | LGQFLLFCHISSHQHDGMEAYVKVD |
| | SCPEEPQLRMKNNEEAEDYDDDLTD |
| | SEMDVVRFDDDNSPSFIQIRSVAKK |
| | HPKTWVHYIAAEEEDWDYAPLVLAP |
| | DDRSYKSQYLNNGPQRIGRKYKKVR |
| | FMAYTDETFKTREAIQHESGILGPL |
| | LYGEVGDTLLIIFKNQASRPYNIYP |
| | HGITDVRPLYSRRLPKGVKHLKDFP |
| | ILPGEIFKYKWTVTVEDGPTKSDPR |
| | CLTRYYSSFVNMERDLASGLIGPLL |
| | ICYKESVDQRGNQIMSDKRNVILFS |
| | VFDENRSWYLTENIQRFLPNPAGVQ |
| | LEDPEFQASNIMHSINGYVFDSLQL |
| | SVCLHEVAYWYILSIGAQTDFLSVF |
| | FSGYTFKHKMVYEDTLTLFPFSGET |
| | VFMSMENPGLWILGCHNSDFRNRGM |
| | TALLKVSSCDKNTGDYYEDSYEDIS |
| | AYLLSKNNAIEPRSFSQNPPVLKRH |
| | QREITRTTLQSDQEEIDYDDTISVE |
| | MKKEDFDIYDEDENQSPRSFQKKTR |
| | HYFIAAVERLWDYGMSSSPHVLRNR |
| | AQSGSVPQFKKVVFQEFTDGSFTQP |
| | LYRGELNEHLGLLGPYIRAEVEDNI |
| | MVTFRNQASRPYSFYSSLISYEEDQ |
| | RQGAEPRKNFVKPNETKTYFWKVQH |
| | HMAPTKDEFDCKAWAYFSDVDLEKD |
| | VHSGLIGPLLVCHTNTLNPAHGRQV |
| | TVQEFALFFTIFDETKSWYFTENME |
| | RNCRAPCNIQMEDPTFKENYRFHAI |
| | NGYIMDTLPGLVMAQDQRIRWYLLS |
| | MGSNENIHSIHFSGHVFTVRKKEEY |
| | KMALYNLYPGVFETVEMLPSKAGIW |
| | RVECLIGEHLHAGMSTLFLVYSNKC |
| | QTPLGMASGHIRDFQITASGQYGQW |
| | APKLARLHYSGSINAWSTKEPFSWI |
| | KVDLLAPMIIHGIKTQGARQKFSSL |
| | YISQFIIMYSLDGKKWQTYRGNSTG |
| | TLMVFFGNVDSSGIKHNIFNPPIIA |
| | RYIRLHPTHYSIRSTLRMELMGCDL |
| | NSCSMPLGMESKAISDAQITASSYF |
| | TNMFATWSPSKARLHLQGRSNAWRP |
| | QVNNPKEWLQVDFQKTMKVTGVTTQ |
| | GVKSLLTSMYVKEFLISSSQDGHQW |
| | TLFFQNGKVKVFQGNQDSFTPVVNS |
| | LDPPLLTRYLRIHPQSWVHQIALRM |
| | EVLGCEAQDLY |
| SEQ ID NO: 3 | DKTHTCPPCPAPELLGGPSVFLFPP |
| Fc region, | KPKDTLMISRTPEVTCVVVDVSHEDP |
| including of | EVKFNWYVDGVEVHNAKTKPREEQY |
| rFVIIIFc and | NSTYRVVSVLTVLHQDWLNGKEYKC |
| certain | KVSNKALPAPIEKTISKAKGQPREP |
| polypeptide | QVYTLPPSRDELTKNQVSLTCLVKG |
| sequences | FYPSDIAVEWESNGQPENNYKTTPP |
| disclosed herein | VLDSDGSFFLYSKLTVDKSRWQQGN |
| comprising Fc not | VFSCSVMHEALHNHYTQKSLSLSPG |
| fused to FVIII or | K |
| any other protein | |
| by a peptide bond | |
| SEQ ID NO: 4 | DKTHTCPPCPAPELLGGPSVFLFPP |
| Processed Fc | KPKDTLMISRTPEVTCVVVDVSHEDP |
| region (not having | EVKFNWYVDGVEVHNAKTKPREEQY |
| a C-terminal | NSTYRVVSVLTVLHQDWLNGKEYKC |
| lysine), including | KVSNKALPAPIEKTISKAKGQPREP |
| of rFVIIIFc and | QVYTLPPSRDELTKNQVSLTCLVKG |
| certain | FYPSDIAVEWESNGQPENNYKTTPP |
| polypeptide | VLDSDGSFFLYSKLTVDKSRWQQGN |
| sequences | VFSCSVMHEALHNHYTQKSLSLSPG |
| disclosed herein | |
| comprising Fc not | |
| fused to FVIII or | |
| any other protein | |
| by a peptide bond | |
| SEQ ID NO: 5 | ATRRYYLGAVELSWDYMQSDLGELP |
| rFVIIIFc | VDARFPPRVPKSFPFNTSWYKKTLF |
| amino acid | VEFTDHLFNIAKPRPPWMGLLGPTI |
| sequence with | QAEVYDTWITLKNMASHPVSLHAVG |
| processed Fc | VSYWKASEGAEYDDQTSQREKEDDK |
| region | VFPGGSHTYVWQVLKENGPMASDPL |
| (not having | CLTYSYLSHVDLVKDLNSGLIGALL |
| a C-terminal | VCREGSLAKEKTQTLHKFILLFAVF |
| lysine) | DEGKSWHSETKNSLMQDRDAASARA |
| | WPKMHTVNGYVNRSLPGLIGCHRKS |
| | VYWHVIGMGTTPEVHSIFLEGHTFL |
| | VRNHRQASLEISPITFLTAQTLLMD |
| | LGQFLLFCHISSHQHDGMEAYVKVD |
| | SCPEEPQLRMKNNEEAEDYDDDLTD |
| | SEMDWRFDDDNSPSFIQIRSVAKKH |
| | PKTWVHYIAAEEEDWDYAPLVLAPD |
| | DRSYKSQYLNNGPQRIGRKYKKVRF |
| | MAYTDETFKTREAIQHESGILGPLL |
| | YGEVGDTLLIIFKNQASRPYNIYPH |
| | GITDVRPLYSRRLPKGVKHLKDFPI |
| | LPGEIFKYKWTVTVEDGPTKSDPRC |
| | LTRYYSSFVNMERDLASGLIGPLLI |
| | CYKESVDQRGNQIMSDKRNVILFSV |
| | FDENRSWYLTENIQRFLPNPAGVQL |
| | EDPEFQASNIMHSINGYVFDSLQLS |
| | VCLHEVAYWYILSIGAQTDFLSVFF |
| | SGYTFKHKMVYEDTLTLFPFSGETV |
| | FMSMENPGLWILGCHNSDFRNRGMT |
| | ALLKVSSCDKNTGDYYEDSYEDISA |
| | YLLSKNNAIEPRSFSQNPPVLKRHQ |
| | REITRTTLQSDQEEIDYDDTISVEM |
| | KKEDFDIYDEDENQSPRSFQKKTRH |
| | YFIAAVERLWDYGMSSSPHVLRNRA |
| | QSGSVPQFKKVVFQEFTDGSFTQPL |
| | YRGELNEHLGLLGPYIRAEVEDNIM |
| | VTFRNQASRPYSFYSSLISYEEDQR |
| | QGAEPRKNFVKPNETKTYFWKVQHH |
| | MAPTKDEFDCKAWAYFSDVDLEKDV |
| | HSGLIGPLLVCHTNTLNPAHGRQVT |
| | VQEFALFFTIFDETKSWYFTENMER |
| | NCRAPCNIQMEDPTFKENYRFHAIN |
| | GYIMDTLPGLVMAQDQRIRWYLLSM |
| | GSNENIHSIHFSGHVFTVRKKEEYK |
| | MALYNLYPGVFETVEMLPSKAGIWR |
| | VECLIGEHLHAGMSTLFLVYSNKCQ |
| | TPLGMASGHIRDFQITASGQYGQWA |
| | PKLARLHYSGSINAWSTKEPFSWIK |
| | VDLLAPMIIHGIKTQGARQKFSSLY |
| | ISQFIIMYSLDGKKWQTYRGNSTGT |
| | LMVFFGNVDSSGIKHNIFNPPIIAR |
| | YIRLHPTHYSIRSTLRMELMGCDLN |
| | SCSMPLGMESKAISDAQITASSYFT |
| | NMFATWSPSKARLHLQGRSNAWRPQ |
| | VNNPKEWLQVDFQKTMKVTGVTTQG |
| | VKSLLTSMYVKEFLISSSQDGHQWT |
| | LFFQNGKVKVFQGNQDSFTPVVNSL |
| | DPPLLTRYLRIHPQSWVHQIALRME |
| | VLGCEAQDLYDKTHTCPPCPAPELL |
| | GGPSVFLFPPKPKDTLMISRTPEVT |
| | CWVDVSHEDPEVKFNWYVDGVEVHN |
| | AKTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |

1. A method of treating a subject with hemophilia A and low bone mineral density (BMD), the method comprising:
(i) selecting a subject having hemophilia A and low BMD, and
(ii) administering to the subject a therapeutically effective amount of a chimeric protein comprising a recombinant FVIII protein and a Fc domain (rFVIIIFc);
wherein administration of the chimeric protein inhibits reduction of BMD in the subject.

2. The method of claim 1, wherein the chimeric protein comprises an amino acid sequence at least 95% identical to an amino acid sequence according to SEQ ID NO: 1.

3. The method of claim 1, wherein the chimeric protein comprises an amino acid sequence at least 95% identical to an amino acid sequence according to SEQ ID NO: 2.

4. The method of claim 1, wherein the chimeric protein comprises an amino acid sequence according to SEQ ID NO: 1.

5. The method of claim 1, wherein the chimeric protein comprises an amino acid sequence at least 95% identical to an amino acid sequence according to SEQ ID NO: 5.

6. The method of claim 1, wherein the chimeric protein comprises an amino acid sequence according to SEQ ID NO: 5.

7. The method of claim 1, wherein the chimeric protein comprises a first polypeptide chain comprising an amino acid sequence at least 95% identical to the amino acid sequence according to SEQ ID NO: 5 and a second polypeptide chain comprising an amino acid sequence at least 95% identical to the amino acid sequence according to SEQ ID NO: 4.

8. The method of claim 1, wherein the chimeric protein comprises a first polypeptide chain comprising an amino acid sequence according to SEQ ID NO: 5 and a second polypeptide chain comprising an amino acid sequence according to SEQ ID NO: 4.

9. The method of claim 8, wherein the first polypeptide chain is covalently bound to the second polypeptide chain via a disulfide bond.

10. The method of claim 9, wherein the first polypeptide chain is covalently bound to the second polypeptide chain via two disulfide bonds in a hinge region of the Fc domain.

11. The method of claim 1 or 10, wherein the chimeric protein is efmoroctocog alfa.

12. The method of any one of claims 1 to 11, wherein the chimeric protein has been produced by human cells.

13. The method of claim 12, wherein the human cells are human embryonic kidney 293 (HEK293) cells.

14. The method of any one of claims 1 to 13, wherein the chimeric protein is administered at a dose of 25-65 IU/kg every 3-5 days.

15. The method of any one of claims 1 to 14, wherein the Fc domain is the Fc domain of human immunoglobulin G1 (IgG1).

16. The method of any one of claims 1 to 15, wherein BMD in the subject is measured by Dual X-Ray Absorptiometry (DXA).

17. The method of any one of claims 1 to 16, wherein the subject is 50 years of age or older.

18. The method of any one of claims 1 to 17, wherein BMD in the subject is determined by T-score.

19. The method of claim 18, wherein the subject is determined to have low BMD if the subject has a T-score of less than −1.0.

20. The method of claim 18, wherein the subject is determined to have low BMD and osteopenia if the subject has T-score between −1.0 and −2.4.

21. The method of claim 18, wherein the subject is determined to have low BMD and osteoporosis if the subject has a T-score of less than −2.5.

22. The method of any one of claims 1 to 16, wherein the subject is younger than 50 years of age.

23. The method of claim 22, wherein BMD in the subject is determined by Z-score.

24. The method of claim 23, wherein the subject is determined to have low BMD if the subject has a Z-score of less than −2.0.

25. The method of any one of claims 1 to 24, wherein the subject is predicted to have low BMD based on the levels of one or more biomarkers of bone formation, bone resorption, and/or bone loss.

26. The method of claim 25, wherein the biomarker is assessed from the peripheral blood or urine of the subject.

27. The method of claim 26, wherein the one or more biomarkers of bone formation comprise bone-specific alkaline phosphatase, procollagen type 1 N-terminal propeptide (P1NP), procollagen type 1 C-terminal propeptide (P1CP), and/or osteocalcin.

28. The method of claim 26, wherein the one or more biomarkers of bone resorption comprise total alkaline phosphatase in serum, the receptor activator of nuclear factor kappa B (RANKL), osteoprotegerin (OPG), tartrate-resistant acid phosphatase (TRAP), hydroxylysine, hydroxyproline, deoxypyridinoline (DPD), pyridinoline (PYD), bone sialoprotein, cathepsin K, tartrate-resistant acid phosphatase 5b (TRAP5b), matrix metalloproteinase 9 (MMP9), and/or C- and/or N-terminal cross-linked telopeptide for type 1 collagen (CTX-1 and NTX-1).

29. The method of any one of claims 1-28, wherein the subject does not have a vitamin D deficiency.

30. The method of any one of claims 1-29, wherein the subject has been previously treated with a Factor VIII without an Fc portion.

31. A method of treating a subject with hemophilia A and an increased risk of fracture, the method comprising:
(i) selecting a subject having hemophilia A and an increased risk of fracture, and
(ii) administering to the subject a therapeutically effective amount of a chimeric protein comprising a recombinant FVIII protein and a Fc domain (rFVIIIFc);
wherein administration of the chimeric protein reduces the risk of fracture in the subject.

32. The method of claim 31, wherein the risk of fracture in the subject is determined by the fracture risk assessment tool (FRAX).

33. The method of claim 32, wherein the risk of fracture in the subject is determined by assessment of low BMD risk factors.

34. The method of claim 33, wherein the low BMD risk factors comprise arthropathy, reduced physical activity, infection with HIV or HCV, vitamin D deficiency, low body mass index (BMI), and/or hypogonadism.

35. A method of reducing the rate of bone mineral density (BMD) loss in a subject, the method comprising:
(i) selecting a subject with low BMD; and
(ii) administering to the subject a therapeutically effective amount of a chimeric protein comprising a coagulation factor and a Fc domain (rFVII1Fc), such that administration of the chimeric protein reduces the rate of BMD loss in the subject.

36. The method of any one of claims 1 to 35, wherein the subject has mild hemophilia A.

37. The method of any one of claims 1 to 35, wherein the subject has moderate hemophilia A.

38. The method of any one of claims 1 to 35, wherein the subject has severe hemophilia A.

39. A method of increasing bone mineral density (BMD) and prophylactically treating bleeding episodes in a subject who has hemophilia A, the method comprising:
(i) identifying a subject who is receiving treatment for hemophilia A with a FVIII protein without an Fc portion, wherein the subject has had adequate blood clotting during the treatment, and wherein the subject has low BMD; and
(ii) discontinuing treatment with the FVIII protein without an Fc portion and administering to the subject a therapeutically effective amount of a chimeric protein comprising a recombinant FVIII protein and a Fc domain (rFVII1Fc),
wherein administration of the chimeric protein increases BMD and prophylactically treats bleeding episodes in the subject.

40. A method of increasing bone mineral density (BMD) and prophylactically treating bleeding episodes in a subject who has hemophilia A, the method comprising:
(i) identifying a subject who is receiving treatment for hemophilia A with a non-factor replacement protein, wherein the subject has had adequate blood clotting during the treatment, and wherein the subject has low BMD; and
(ii) discontinuing treatment with the non-factor replacement protein and administering to the subject a therapeutically effective amount of a chimeric protein comprising a recombinant FVIII protein and a Fc domain (rFVII1Fc),
wherein administration of the chimeric protein increases BMD and prophylactically treats bleeding episodes in the subject.

41. A method of increasing bone mineral density (BMD) and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and low BMD, and wherein administration of the chimeric protein increases BMD and prophylactically treats bleeding episodes in the subject.

42. A method of reducing risk of fracture and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and an increased risk of fracture, and wherein administration of the chimeric protein reduces the risk of fracture and prophylactically treats bleeding episodes in the subject.

43. A method of reducing rate of bone mineral density (BMD) loss and prophylactically treating bleeding episodes in a subject, the method comprising administering to the subject a therapeutically effective amount of a chimeric protein comprising a recombinant FVIII protein and a Fc domain (rFVII1Fc), wherein the subject has been identified as having hemophilia A and BMD loss, and wherein administration of the chimeric protein reduces the rate of BMD loss and prophylactically treats bleeding episodes in the subject.

44. A method of increasing bone mineral density (BMD) and prophylactically treating bleeding episodes in a subject who has hemophilia A and is being treated with a FVIII protein without an Fc portion, the method comprising discontinuing treatment with the FVIII protein without an Fc portion and administering to the subject a therapeutically effective amount of a chimeric protein comprising a recombinant FVIII protein and a Fc domain (rFVII1Fc), wherein the subject has been identified as having low BMD and adequate blood clotting during treatment with the FVIII protein without an Fc portion, and wherein administration of the chimeric protein increases BMD and prophylactically treats bleeding episodes in the subject.

45. A method of increasing bone mineral density (BMD) and prophylactically treating bleeding episodes in a subject who has hemophilia A and is being treated with a non-factor replacement protein, the method comprising discontinuing treatment with the non-factor replacement protein and administering to the subject a therapeutically effective amount of a chimeric protein comprising a recombinant FVIII protein and a Fc domain (rFVII1Fc), wherein the subject has been identified as having low BMD and adequate blood clotting during treatment with the non-factor replacement protein, and wherein administration of the chimeric protein increases BMD and prophylactically treats bleeding episodes in the subject.

46. The method of any one of claims 1-45, wherein the subject has been previously treated to reduce bleeding associated with hemophilia A using a Factor VIII protein without an Fc portion.

47. The method of any one of claim 39, 44, or 46, wherein the Factor VIII protein without an Fc portion is PEGylated FVIII that is not fused to a Fc domain.

48. The method of any one of claim 39, 44, or 46, wherein the Factor VIII protein without an Fc portion is single-chain FVIII that is not fused to a Fc domain.

49. The method of any one of claim 39, 44, or 46, wherein the Factor VIII protein without an Fc portion is recombinant FVIII that does not comprise a moiety that extends half-life thereof in humans.

50. The method of any one of claim 39, 44, or 46, wherein the Factor VIII protein without an Fc portion is blood-derived FVIII or plasma-derived FVIII.

51. The method of any one of claim 39, 44, or 46, wherein the Factor VIII protein without an Fc portion is damoctocog alfa pegol, turoctocog alfa pegol, turoctocog alfa, lonoctocog alfa, simoctocog alfa, rurioctocog alfa pegol, or octocog alfa.

52. The method of any one of claims 1-51, wherein the subject has been previously treated to reduce bleeding associated with hemophilia A using a non-factor replacement protein.

53. The method of claim 52, wherein the non-factor replacement protein is emicizumab.

54. The method of claim 53, wherein the emicizumab is emicizumab-kxwh.

55. The method of any one of claim 30 or 36-54, wherein the subject had adequate blood clotting during treatment with the Factor VIII protein without an Fc portion or the non-factor replacement protein.

56. The method of any one of claims 1-55, wherein the subject has low BMD at a bone site and/or joint where bleeding has not been detected.
